(19) United States
(12) Patent Application Publication
(10) Pub. No.: US 2006/0183696 A1
(43) Pub. Date: Aug. 17, 2006

(54) Use of immune cell specific conjugates for treatment of inflammatory diseases of gastrointestinal tract

(75) Inventors: Mladen Mercep, Zagreb (HR); Milan Mesic, Zagreb (HR); Linda Tomaskovic, Zagreb (HR); Stribor Markovic, Zagreb (HR)

Correspondence Address: DARBY & DARBY P.C., P. O. BOX 5257, NEW YORK, NY, 10150-5257, US

(73) Assignee: Pliva-Istrazivacki Institut d.o.o., Zagreb (HR)

(21) Appl. No.: 11/355,808

(22) Filed: Feb. 15, 2006

Related U.S. Application Data
(63) Continuation-in-part of application No. 11/201,685, filed on Aug. 10, 2005.
(60) Provisional application No. 60/601,087, filed on Aug. 12, 2004.
(60) Provisional application No. 60/603,315, filed on Aug. 19, 2004.

(51) Int. Cl. A61K 31/7048; C07J 17/00; C07H 17/08

(52) U.S. Cl. 514/26; 536/7.4; 536/6.1

(57) ABSTRACT

The present invention is directed to methods for the prevention and treatment of inflammatory diseases, disorders, and conditions of gastrointestinal tract by administering to a patient in need of such treatment, conjugate compounds of Formula VII having low oral-bioavailability, or pharmaceutically acceptable salts, prodrugs, or solvate thereof: 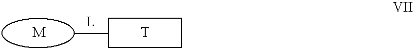 wherein M represents a macrolide subunit possessing the property of accumulation in inflammatory cells, T represents an anti-inflammatory subunit that can be a steroid or nonsteroid (nonsteroidal moiety) derived from a non-steroid drug with anti-inflammatory, analgesic and/or antipyretic activity (NSAID) and L represents a linker covalently linking M and T. The present disclosure is also directed to pharmaceutical compositions containing conjugate compounds of Formula VII having low oral-bioavailability.

This application claims benefit of U.S. patent application Ser. No. 11/201,685 filed Aug. 10, 2005, U.S. Provisional Appl. 60/601,087 filed Aug. 12, 2004, and U.S. Provisional Appl. 60/603,315 filed Aug. 19, 2004, each of which are herein incorporated by reference in their entirety.

### FIELD OF THE INVENTION

The present invention relates to the use for the treatment and prevention of inflammatory diseases, disorders, and conditions of the gastrointestinal tract in a subject of a conjugate or its pharmaceutically acceptable salt, prodrug or solvates which has low bioavailability when administered orally or when otherwise delivered to the gastrointestinal mucosa of a conjugate of (i) a macrolide subunit which preferentially accumulates in immune system cells and (ii) an anti-inflammatory drug. The anti-inflammatory drugs can be selected from one or more of anti-inflammatory corticosteroids or nonsteroidal anti-inflammatory drugs (NSAIDs). Specifically, the low orally-bioavailable conjugate compounds of the invention are represented by formula VII below. In particular, this invention relates to the long-term maintenance treatment of inflammatory bowel diseases which are in remission totally or partially and which respond to treatment with glucocorticoids or NSAIDs (e.g., Crohn's disease, ulcerative colitis, and celiac disease).

### BACKGROUND OF THE INVENTION

Human inflammatory bowel disease (IBD) is generally described as a condition that occurs in genetically susceptible individuals because of an aberrant immune response to enteric antigens. It is thought that development of IBD results from a failure of the mucosal immune system to attenuate the response to endogenous antigens. IBD is a family of chronic, relapsing, and tissue-destructive diseases characterized by dysfunction of mucosal T cells, abnormal cytokine production, and cellular inflammation that ultimately leads to damage of intestinal mucosa. Clinically, IBD encompasses Crohn's disease (CD) and ulcerative colitis (UC) (Bamford K. D., FEMS Immunol. Med. Microbiol., 1999, 24:161-8; Mayer et al. Current concept of IBD: Etiology and pathogenesis. In “Inflammatory Bowel Disease”, 5th edition 2000, Kirsner J B editor. W. B. Saunders Company, pp 280-296).

Crohn's disease is a chronic inflammatory disease of unknown etiology that can affect any part of the bowel. Although lesions may start superficially, the inflammatory process extends thorough the bowel wall to draining lymph nodes. The course of the disease may be relapsing or continuous, mild or severe. It cannot be cured by resection of the involved segment of the bowel. Most patients with Crohn's disease undergo surgery at some point, but subsequent relapse is common and continuous medical treatment is the norm.

Ulcerative colitis (UC) is also a chronic inflammatory disease of unknown etiology but it affects only the large bowel and is limited to the bowel mucosa except in very severe cases. The course of this disease may also be relapsing or continuous, mild or severe. It can be cured by total colectomy, which may be necessary for acute severe disease or chronic unremitting disease cases. Most patients with ulcerative colitis are managed medically rather than surgically.

For treatment of severe Crohn's disease, glucocorticoid steroids are the treatment of choice, but only until remission is achieved, after which they should be discontinued. If the disease does not satisfactorily remit, glucocorticoids are used to maintain control of symptoms. Sulfasalazine is used in less severe cases, especially if the disease involves the colon. For symptomatic treatment of Crohn's disease, analgesics for pain and opiates for diarrhea control are also used. However, many patients eventually require surgery.

For the treatment of acute attacks of ulcerative colitis, glucocorticoid steroids (prednisolone, prednisolone acetate or budesonide) are used (Mulder C J and G N Tytgat, Aliment. Pharmacol. Ther. 1993, 7:125-30). After remission has been achieved, UC is also treated with sulfasalazine for maintenance of remission. Sulfasalazine has many side effects due to absorption of the sulfapyridine moiety from the colon (Smolen J. S. et al, The Lancet, 1999; 353:259-266). New drugs have recently been developed like 5-amino-salicylic acid or compounds having a 5-aminosalicylic acid moiety, and are as effective as sulfasalazine but without the side effects associated with sulfapyridine class of drugs. However, these new drugs do have their own side effects, such as diarrhea (Ardizzone S and G B Porro, Drug Saf. 2002, 25:561-82).

Celiac disease is a chronic intestinal disease that represents reaction to gluten present in wheat and rye proteins, and leads to changes in the small intestinal mucosa and impaired absorption of nutrients in patients who do not tolerate gluten. Although not known to be related to IBD, it has certain symptomatology in common, e.g., local infiltration of inflammatory cells, malabsorption and malnutrition due to colonic tissue damage, elevated mediators of inflammation in the local tissue. Many patients today are treated with a gluten-free diet high in proteins and normal in fat. However, some patients do not respond to such treatment. These patients are treated with glucocorticoids such as hydrocortisone, prednisone or prednisolone. This treatment has to be eventually discontinued due to the fact that chronic administration of such doses of systemically active steroids runs a significant risk of causing severe side effects. Since these are diseases with acute relapses that can occur after long periods (several years) of remission in which no clinical signs are present, using maintenance therapy involving the administration of unmodified steroids cannot be justified.

International Publication No. WO 02/055531 A1, herein incorporated by reference in its entirety, discloses conjugate compounds represented by the Formula I: 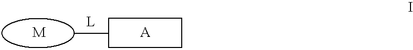
wherein M represents a macrolide subunit possessing the property of accumulation in inflammatory cells, A represents an anti-inflammatory subunit that can be steroid or nonsteroid, and L represents a linker molecule linking M and A, (b) their pharmacologically acceptable salts, prodrugs and solvates, (c) processes and intermediates for their preparation, and (d) their use in the treatment of inflammatory diseases and conditions in humans and animals. In WO 02/05531, the conjugate steroid-macrolide compounds are mostly linked with the steroid subunit at the N/9a-position of macrolide ring.

U.S. Published Application 2004 0014685 herein incorporated by reference in its entirety relates to compounds represented by Formula II. 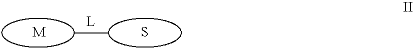
wherein M represents a macrolide subunit (macrolide moiety) derived from macrolide possessing the property of accumulation in inflammatory cells, S represents a steroid subunit derived from a steroid drug with anti-inflammatory activity and L represents a linker molecule linking M and S to their pharmaceutically acceptable salts and solvates processes and intermediates for their preparation and to their use in the treatment of inflammatory diseases and conditions in humans and animals. US Published Application 20040077612 herein incorporated by reference in its entirety relates to new compounds represented by Formula III. 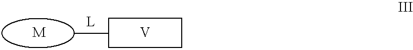
wherein M represents a macrolide subunit (macrolide moiety) derived from macrolide possessing the property of accumulation in inflammatory cells, V represents an anti-inflammatory steroid or non steroid subunit or an anti neoplastic or antiviral subunit and L represents a linking group covalently linking M and V to their pharmaceutically acceptable salts and solvates processes and intermediates for their preparation and to their use in the treatment of inflammatory diseases and conditions in humans and animals.

US Published Application 2004 0097434 herein incorporated by reference in its entirety relates to new compounds represented by formula IV. 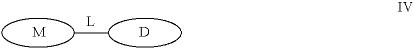
wherein M represents a macrolide subunit (macrolide moiety) derived from macrolide possessing the property of accumulation in inflammatory cells, D represents a nonsteroidal subunit (nonsteroidal moiety) derived from a nonsteroid drug with anti-inflammatory, analgesic and/or antipyretic activity (NSAID) and L represents a linking group covalent linking M and D to their pharmaceutically acceptable salts and solvates processes and intermediates for their preparation and to their use in the treatment of inflammatory diseases and conditions in humans and animals.

International Publication No. WO 04/005310 A2, herein incorporated by reference in its entirety, describes conjugate steroid-macrolide compounds of Formula V:

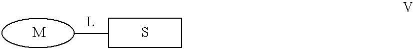

wherein symbol M in the above structure represents a macrolide subunit possessing the property of accumulation in inflammatory cells, S represents an anti-inflammatory steroid subunit and L represents a linker covalently linking M and S. WO 04/005310 A2 describes conjugate steroid-macrolide compounds of Formula II having the steroid subunit linked through the 17α-OH group with the macrolide subunit at the position C/11 or N/9a of a macrolide having a modified or eliminated dimethylamino group of desosamine sugar; and also conjugate steroid-macrolide compounds with the macrolide subunit linked to the steroid through position C/6 of the macrolide lactonic ring, or through position C/4″ of the cladinose sugar, or through the amine functionality at position C/3′ of the desosamine sugar.

International Publication No. WO 04/005309, herein incorporated by reference in its entirety, discloses (a) new conjugate compounds represented by Formula VI: 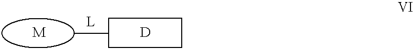
wherein M represents a macrolide subunit (macrolide moiety) derived from macrolide possessing the property of accumulation in inflammatory cells, D represents a nonsteroidal subunit (nonsteroidal moiety) derived from a non-steroid drug with anti-inflammatory, analgesic and/or antipyretic activity (NSAID) and L represents a linking group covalently linking M and D; (b) their pharmacologically acceptable salts, prodrugs and solvates, (c) processes and intermediates for their preparation, and (d) their use in the treatment of inflammatory diseases and conditions in humans and animals.

U.S. Published Application 2005 008003, herein incorporated by reference in its entirety, describes yet further conjugate compounds having a steroid or non-steroidal anti-inflammatory subunit D linked via the chain L to position N/9a of an aglycone type macrolide subunit.

Patent Application Serial Number PCT/IB2005/003213 herein incorporated by reference in its entirety, describes yet further conjugate compounds having a steroid or non-steroidal anti-inflammatory subunit Z linked via the chain L to position 11 of an aglycone type macrolide subunit and further conjugate compounds having a aglycone type macrolide subunits M linked via the chain L to position 17alpha of a steroid subunit.

The use of conjugates having low bioavailability when administered by oral or enteral route where the conjugates comprises (i) an immune cell specific macrolide compound combined with (ii) an anti-inflammatory corticosteroids or nonsteroidal anti-inflammatory drugs (NSAIDs) and pharmaceutically acceptable salts, prodrugs and solvates thereof for the maintenance treatment and prevention of recurrence of inflammatory diseases, disorders, and conditions of the gastrointestinal tract has hitherto not been described.

### SUMMARY OF THE INVENTION

The present invention is directed to methods for the prevention of recurrence and for the treatment (including not only acute-phase treatment but also maintenance treatment) of inflammatory diseases, disorders, and conditions of the gastrointestinal tract by administering to a patient in need of such treatment a conjugate compound according to Formula VII, or pharmaceutically acceptable salts, prodrugs, or solvates thereof having low oral bioavailability: 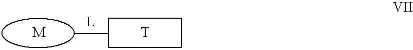
wherein M represents a macrolide subunit possessing the property of accumulation in inflammatory cells, T represents an anti-inflammatory subunit that can be a steroid or a nonsteroid (nonsteroidal moiety) derived from a non-steroid drug with anti-inflammatory, analgesic and/or antipyretic activity (NSAID) and L represents a linker covalently linking M and T, and continuing said administration into or through the maintenance phase of treatment of such disorders. In other words, while some of these NSAID compounds (in unmodified form) may have been proposed for use in treating these inflammatory conditions of the gut their use was contemplated as substitutes for unconjugated corticosteroids. Accordingly, such use would be limited to the acute phase of the disease and would not be extended to long-term maintenance therapy or to long-term use for the prevention of recurrence of the disease. If the use of unmodified NSAIDs was continued, there was increased risk of gastrointestinal damage occurring from the use of NSAIDs. The foregoing conjugate compounds of the present invention, however, could be substitutes for standard glucocorticoid therapy and could be used over the long term without (or with reduced) risk of GI side effects such as those associated with long term use of unconjugated NSAIDs. The NSAID conjugates of the present invention by not being substantially absorbed are suitable for long term maintenance therapy in preventing or delaying relapse. Additionally, the modified steroid conjugates of the present invention are also not substantially absorbed, unlike standard glucocorticoids, and thus they show systemic effect and be appropriate for long term therapy and thus useful for maintenance therapy to prevent or delay relapse. The present compounds are also less likely to damage the intestinal mucosa in a way characteristic of NSAIDs especially those used in long-term therapy, which are known to cause gastrointestinal ulceration and renal and respiratory toxicity. The undesirable side effects of unmodified NSAIDs have been attributed to the inhibition of prostaglandins in the affected organ, i.e., the gastrointestinal mucosa.

### BRIEF DISCRIPTION OF THE DRAWINGS

FIG. 1A is a bar graph showing macroscopic damage score after administration to a rat model of compounds 1′, 2′ and 3′ or budesonide (positive control) or vehicle (negative control) to measure damage to the colon induced by TNBS. Significance of results was analyzed using Mann-Whitney test.

FIG. 1B is a bar graph showing histological damage score after administration to a rat model of compounds 1′, 2′ and 3′ or budesonide (positive control) or vehicle (negative control) to measure damage to the colon induced by TNBS. Significance of results was analyzed using Mann-Whitney test.

FIG. 2 is a bar graph showing macroscopic damage score after administration of varying dosages of compound 1′ or budesonide (positive control) or vehicle (negative control) to measure damage to the colon induced by TNBS. Significance of results was analyzed using Mann-Whitney test.

FIG. 3 is a bar graph showing the colonic macroscopic damage score/colonic ulceration for Compounds 1, 2, and 3 as well as for a native control, vehicle control, sulfasalazine, and budesonide.

### DETAILED DESCRIPTION OF THE INVENTION

The present invention solves the problem of effective prevention and treatment of inflammatory diseases, disorders, and conditions of the gastrointestinal tract, including, but not limited to, irritable bowel disease (IBD) by use of conjugate compounds of the Formula VII that exhibit low bioavailability when administered orally. The compounds of Formula VII that exhibit low oral bioavailability exhibit fewer side effects than standard glucocorticoids, since they are not absorbed from the gastrointestinal tract to systemic circulation. This is an advantage over the use of standard steroids. The low orally-bioavailable M-L-T conjugates of Formula VII of the present invention reach the inflamed site in the bowel where they exert an anti-inflammatory effect, but they do not cause systemic side effects since they are not absorbed. Therefore, the conjugates of the Formula IV which exhibit low oral bioavailability can safely be used not only in acute phases of the disease but also, and more importantly, during maintenance therapy of IBD, when the use of conventional steroids and conjugates of high oral bioavailability would not be indicated or would not be treatment of choice because of the long duration of the therapy. The maintenance phase of therapy begins when the disease is in partial or total remission. Conventional therapy of acute phase, such as acute relapse lasts 4-6 weeks if 5-aminosalicylic acid and its derivatives is used as the therapeutic agent, and 2-3 weeks if steroids like prednisone or hydrocortisone are used. Responsiveness to treatment is ascertained by laboratory tests (like stool examination and blood analysis) so effectiveness is ascertained after treatment has been initiated. Unconjugated NSAIDs are not indicated for IBD.

Accordingly, the present invention provides a method for the prevention and treatment of inflammatory diseases, disorders, and conditions of the gastrointestinal tract, which comprises administering to a patient in need thereof an effective amount of a conjugate compound of Formula VII that exhibits low bioavailability when administered by the oral route, or a pharmaceutically acceptable salt, prodrug or solvate thereof.

The present invention also provides a method for the prevention or delay of recurrence (including maintenance phase treatment) of inflammatory bowel diseases which respond to treatment with glucocorticoids, such as, but not limited to, Crohn's disease, ulcerative colitis, and celiac disease, by administering to a patient an effective amount of a conjugate compound of the Formula VII which exhibits low oral-bioavailability, or a pharmaceutically acceptable salt, prodrug or solvate thereof.

In one embodiment of the present invention the conjugate compound of Formula VII, or salt, prodrug, or solvate thereof exhibits less than 10% oral bioavailability, i.e., between about 0% and about 10%.

In another embodiment of the present invention the conjugate compound of Formula VII, or salt, prodrug, or solvate thereof exhibits less than 5% oral bioavailability, i.e., between about 0% and about 5%.

In yet another embodiment of the present invention the conjugate compound of Formula VII, or salt, prodrug, or solvate thereof exhibits less than 2% oral bioavailability, i.e., between about 0% and about 2%.

The present invention is also directed to pharmaceutical compositions comprising at least one compound selected from conjugates of the general Formula VII which exhibit low oral-bioavailability, and pharmaceutically acceptable salts, prodrugs or solvates thereof, and a pharmaceutically acceptable carrier.

In a specific embodiment, the composition comprises at least one compound selected from the group of conjugates of Formula VII which exhibits less than 10% oral bioavailability. In another specific embodiment, the composition comprises at least one compound selected from the group of conjugates of Formula VII which exhibits oral bioavailability of less than 5%. In a further specific embodiment, the composition comprises at least one compound selected from the group of conjugates of Formula VII which exhibit oral bioavailability of less than 2%.

A particular characteristic of this conjugates is their low bioavailability, which is due to very high molecular mass of such molecules (>700). According to the “rule of five” (Lipinski C. A. et al., Adv. Drug. Deliv. Rev., 2001, 46:3-26) which is followed by majority of drugs, molecular mass of orally bioavailable drugs should be less then 500. There are few successful orally active drugs that fail to fit this rule. Additionally, bioavailability of conjugates of Formula VII could be estimated on the basis of Caco-2 in vitro model which is accepted as fairly good model for prediction of oral bioavailability (Yee S and W. W. Day, Applications of Caco-2 cells in drug discovery and development. In “Handbook of drug metabolism”, 1999, Woolf T. F. editor, Marcel Dekker Inc., pp 507-522). In addition, a prerequisite for oral absorption in that drug must be present in solution at the absorption site. Only vesicular absorption or pynocytosis does not require it. Since conjugates of Formula VII have low aqueous solubility, it is predicted that they would not be absorbed from gut by traditional mechanisms that require compound solubility (trancellular and paracellular diffusion plus carrier-mediated transport). At the same time, pynocytosis and/or vesicular absorption would enable their penetration into the gut wall and good local anti-inflammatory activity.

Particularly, the invention provides an oral pharmaceutical composition which comprises at least one compound selected from the group of conjugates of Formula VII their pharmaceutically acceptable salts, prodrugs or solvates, which exhibits oral from about 0% to about 10%, preferably from about 0% to about 5%, most preferably from about 0% to about 2%, for use in human and veterinary medicine, for the delay or prevention of recurrence and for the maintenance phase treatment of inflammatory bowel diseases, including those that respond to treatment with glucocorticoids, such as, but not limited to, Crohn's disease, ulcerative colitis, and celiac disease.

The Conjugates of Formula VII

In one aspect of the present invention, the low orally-bioavailable conjugates of Formula VII, and pharmaceutically acceptable salts, prodrugs, and solvates thereof, are represented as shown below: 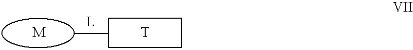
wherein M represents a macrolide subunit selected from the group consisting of 12-, 14-, 15-, 16-, 17-, and 18-membered lactonic ring molecules wherein “membered” refers to the number of carbon atoms or heteroatoms in the lactonic ring said macrolide having the property of accumulating within mammalian immune system cells that mediate inflammatory immune responses, T represents an anti-inflammatory subunit that can be a steroid or nonsteroid (nonsteroidal moiety) derived from a non-steroid drug with anti-inflammatory, analgesic and/or antipyretic activity (NSAID) and L represents a linker covalently linking M and T.

In preferred embodiments, this invention relates to the use of compounds, represented by the Formula VII, and salts, prodrugs and solvates thereof, wherein M specifically represents a 14- or 15-member lactonic ring macrolide subunit most preferably represented by the Formula VIII:

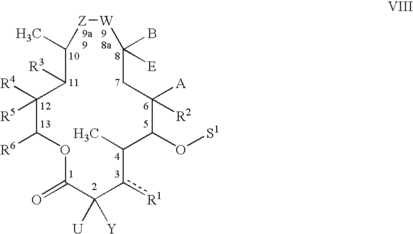

wherein

(i) Z and W independently are 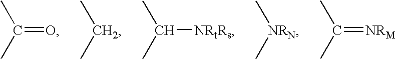
, or a bond, wherein
Rₜ and Rₛ independently are H or alkyl (preferably methyl or H);
R_{M} is OH, OR^{p}, alkoxy or substituted alkoxy (in either Syn or Anti configurations or mixtures thereof)
R_{N} is H, R^{p}, alkyl, alkenyl, alkynyl, alkoxy, alkoxyalkyl, or —C(═X)—NRₜRₛ; and
X is O or S;

provided that Z and W cannot both simultaneously be 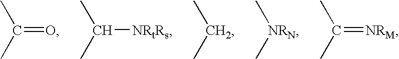
or a bond,

(ii) U and Y are independently H, halogen, alkyl, or hydroxyalkyl (preferably H, methyl, or hydroxymethyl);

(iii) R¹ is hydroxy, OR^{p}, —O—S², or ═O;

(iv) S¹ is H or a sugar moiety at position C/5 (e.g., a desozamine group) of the formula:

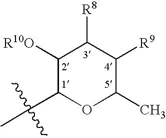

wherein

R⁸ and R⁹ are both hydrogen or together form a bond, or R⁹ is hydrogen and R⁸ is —N(CH₃)R^{y}, wherein

R^{y} is R^{p}, R^{z} or —C(═O)R^{z}, wherein R^{z} is hydrogen or cycloalkyl (preferably cyclohexyl) or alkyl (preferably a C₁-C₇ alkyl) or alkenyl (preferably C₂-C₇-alkenyl) or alkynyl (preferably C₂-C₇-alkynyl) aryl or heteroaryl or alkyl substituted with C₂-C₇ alkyl, C₂-C₇ alkenyl, C₂-C₇ alkynyl, aryl or heteroaryl (R^{y} is preferably hydrogen, methyl, ethyl, n-propyl, i-propyl, n-butyl, —C(═O)CH₃, —CH₂-phenyl, or cyclohexyl);

R¹⁰ is hydrogen or R^{p};

(v) S² is H or a sugar moiety (e.g., is a cladinosyl group) of the formula

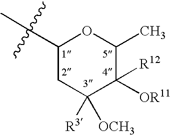

wherein R^{3′} can be H or methyl and R¹¹ is hydrogen or R^{p} or O—R¹¹ is a group that with R¹² and with C/4″ carbon atom forms a >C═O or epoxy group;

R¹² is hydrogen, alkyl, alkyl-R^{p}, R^{p}, or a group that with O—R¹¹ and with C/4″ carbon atom forms a >C═O or epoxy group;

(vi) R² is H, hydroxy, OR^{p} group, alkoxy (preferably C₁-C₄ alkoxy, most preferably methoxy) or substituted alkoxy;

(vii) A is H or methyl;

(viii) B is methyl or epoxy;

(ix) E is H or halogen (preferably fluorine);

(x) R³ is hydroxy, OR^{p} group or alkoxy (preferably C₁-C₄ alkoxy, most preferably methoxy), substituted alkoxy or R³ is a group that can combine with R⁵ to form a “bridge” (e.g., a cyclic carbonate or carbamate) or if W or Z is 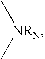
R³ is a group that can combine with W or Z to form a “bridge” (e.g., a cyclic carbamate);

(xi) R⁴ is C₁-C₄ alkyl (preferably methyl);

(xii) R⁵ is H, hydroxy, OR^{p} group, C₁-C₄ alkoxy, substituted alkoxy or a group that may combine with R³ to form a bridge (e.g., a cyclic carbonate or carbamate);

(xiii) R⁶ is H or C₁-C₄ alkyl (preferably methyl or ethyl);

wherein the subunit M has a linkage site through which it is linked to the subunit T via the linking group L, the linkage site being at one or more of the following:

a) any reactive hydroxy, nitrogen, or epoxy group located on macrolide ring, sugar moiety S¹, sugar moiety S², or an aglycone oxygen or nitrogen when S¹ and/or S² is cleaved off;

b) a reactive >N—R_{N} or —NRₜRₛ or 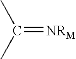
group located on Z or W;

c) a reactive hydroxy group located at any one of R¹, R², R³, and R⁵;

d) any other group that can be first derivatized to a hydroxy or —NRₜRₛ group and then linked to K (e.g., OH→═O→epoxy→ 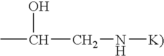
wherein K is the part of the linking molecule L.

One or more R^{p} groups may be independently present in the macrolide subunit of Formula VIII, wherein R^{p} represents a protective group which may be selected from alkyl (preferably methyl), alkanoyl (preferably acetyl), alkoxycarbonyl (preferably methoxycarbonyl or tert-butoxycarbonyl), arylmethoxycarbonyl (preferably benzyloxycarbonyl), aroyl (preferably benzoyl), arylalkyl (preferably benzyl), alkylsilyl (preferably trimethylsilyl) or alkylsilylalkoxyalkyl (preferably trimethylsilylethoxymethyl).

Also preferred are semiglycone compounds according to formula VIII where R¹ is hydroxyl and S¹ is a sugar moiety of the formula (R¹⁰, R⁸, R⁹ are as previously defined):

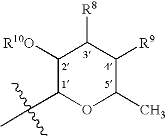

Also preferred are semiglycone compounds according to formula VIII where S¹ is H and R¹ is O—S² where S² is a sugar moiety of the formula (R³, R¹¹ and R¹² are as previously defined):

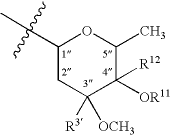

Also preferred are aglycone compounds according to formula VIII is wherein S¹ is hydrogen and R¹ is hydroxyl.

The Linker L

L can be selected to be a linking group represented by the Formula IXA or IXB:
X¹—(CH₂)ₘ—X² or IXA
X¹—(CH₂)ₘ-Q-(CH₂)ₙ—X² IXB

wherein

X¹ is selected from: —CH₂, —CH₂—NH—, —C(═O)—, —OC(═O)—, ═N—O—, —OC(═O)NH— or

—C(═O)NH—;

X² is selected from: —NH—, —CH₂—; —NHC(═O)—, —C(═O)—, —O— or —OC(═O)—;

Q is —NH— or —CH₂—;

wherein each —CH₂— or —NH— group is optionally substituted by C₁-C₇-alkyl, C₂-C₇-alkenyl, C₂-C₇-alkynyl, C(═O)R^{x}, C(═O)OR^{x}, C(═O)NHR^{x} wherein R^{x} may be C₁-C₇-alkyl, aryl or heteroaryl;

the symbols m and n are independently a whole number from 0 to 8

with the proviso that if Q=NH; n cannot be zero.

The foregoing definition of the linking group is preferred not only for conjugates of NSAIDS and macrolides of Formula VIII but for any conjugate within Formula VII. Other linking groups can be used as long as they provide the necessary spacer. Preferred linker molecules are those having a length of 1-20 carbon atoms (not counting heteroatoms in the chain) and those having a length of 2-10 carbon atoms are most preferred. Preferably, the llinkage of L to the macrolide moiety is effected either through the ring nitrogen atom at position 9a or through the hydroxy group at position 11 and position 6 or through the 2′ hydroxy or the 3′ amino group of the desosamine sugar moiety or through the 4″ hydroxy group of the cladinose sugar or if the M moiety is aglycone or missing one of the desosamine and cladinose sugar groups, through the OH group created at position 5 or 3 of the macrolide ring. The linker can serve to link one subunit of the Formula VII, e.g., M, with another subunit of Formula VII, e.g., T, as is well-known in the art. See, e.g., U.S. Pat. No. 6,297,260, which is incorporated by reference in its entirety, especially its claim 1 and the specific list of NSAIDs moieties recited herein and the spacers or linkers used to attach them to the nitrogen oxide group.

The Subunit T

In one embodiment, T represents a nonsteroidal subunit derived from a nonsteroidal anti-inflammatory drug (NSAID) or a steroid subunit that can be represented by the substructure IX:

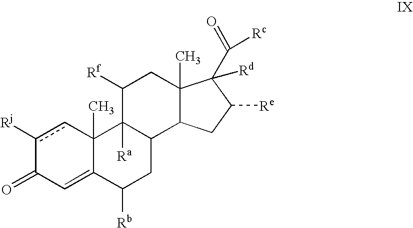

wherein

R^{a} and R^{b} are, independently of each other, hydrogen or halogen;

R^{c} is hydroxy, alkoxy (preferably methoxy), substituted alkoxy, alkyl, thiocarbamoyl, carbamoyl or a valence-bond attached to X² of chain L;

R^{d} and R^{e} are, independently of each other, hydrogen, hydroxy, methyl or C₁-C₄ alkoxy (preferably methoxy or n-propoxy) or each are a group that forms a 1,3-dioxolane ring with the other (optionally alkyl or alkenyl mono- or di-substituted) (preferably a 2,2-dimethyl or 2-monopropyl or trans-propenyl ring) or a valence bond attached to X² of chain L;

R^{f} is hydrogen, hydroxy, chlorine, or ═O forming a keto group with the carbon atom it is attached to;

R^{j} is hydrogen or chlorine

and their pharmacologically acceptable salts, prodrugs, and solvates.

Also encompassed within the present invention are steroid subunits disclosed in WO 94/14834, incorporated herein in its entirety by reference, wherein the group >CH—C(═O)—R^{c} is replaced by the group >CH—S(O)ₙ—R^{c}, wherein n is an integer of 0 to 2. See WO 94/14834, especially pp 2-3.

More generally, steroids useful as a source of steroid subunits in the present invention include, but are not limited to, corticosteroids (such as glucocorticoids and mineralocorticoids) and androgens. Non-limiting examples of corticosteroids include cortisol, cortisone, clobetasol, hydrocortisone, fludrocortisone, fludroxycortide, flumetasone, flunisolide, fluocinolone, fluocinonide, fluocortolone, fluorometholone, prednisone, prednisolone, 6-alpha-methylprednisolone, triamcinolone, alclometasone, beclometasone, betamethasone, budesonide, dexamethasone, amcinonide, cortivazol, desonide, desoximethasone diflucortolone, difluprednate, fluclorolone, dichlorisone, fluperinidene, fluticasone, halcinonide, meprednisone, methylprednisolone, paramethasone, prednazoline, prednylidene, tixocortol, triamcinolone, and acid derivatives thereof, e.g., acetate, propionate, dipropionate, valerate, phosphate, isonicotinate, metasulfobenzoate, tebutate, and hemisuccinate.

In Formula VII the expression “nonsteroidal subunits” denotes subunits derived from nonsteroidal anti-inflammatory drugs (NSAIDs) and other drugs with anti-inflammatory, anti-allergic and immunosuppressive properties, such as aceclofenac, acemetacin, acetaminophen, acetaminosalol, acetyl-salicylic acid, acetyl-salicylic-2-amino-4-picoline-acid, 5-aminoacetylsalicylic acid, alclofenac, amino-profen, amfenac, anileridine, azathioprine, bendazac, benoxaprofen, bermoprofen, α-bisabolol, bromfenac, 5-bromosalicylic acid acetate, bromosaligenin, bucloxic acid, butibufen, carprofen, CC 1088 (Celgene), CC 5013 (Celgene), CDC 801 (Celgene), celecoxib, chromoglycate, cinmetacin, cipamfylline (GlaxoSmithKline), clindanac, clopirac, COX-189 (Novartis), cyclosporine, sodium diclofenac, diflunisal, ditazol, enfenamic acid, etodolac, etofenamate, etoricoxib (Merck), felbinac, fenbufen, fenclozic acid, fendosal, fenoprofen, fentiazac, fepradinol, FK-506, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, JTE-522 (Japan Tobacco Inc.), ketoprofen, ketorolac, L-745337 (Merck), leflunomide, lornoxicam, loxoprofen, meclofenamic acid, mefenamic acid, meloxicam, mesalamine, mesalazine, methotrexate, metiazinic acid, mofezolac, montelukast, mycophenolic acid, naproxen, niflumic acid, olsalazine, oxaceprol, oxaprozin, oxyphenbutazone, parsalmide, perisoxal, phenyl-acethyl-salicylate, phenylbutazone, phenylsalicylate, teophyline, pyrazolac, piroxicam, pirprofen, pranoprofen, protizinic acid, rapamycine, rofecoxib, salacetamide, salicylamide-O-acetyl acid, salicylsulphuric acid, salicin, salicylamide, salsalate, sulindac, sulfasalazine, suprofen, suxibutazone, tenoxicam, thalidomide, tetrafluorthalidomide, tiaprofenic acid, tiaramide, tinoridine, tolfenamic acid, tolmetin, tomoxiprol, tropesin, valdecoxib (Searle), xenbucin, ximoprofen, zaltoprofen, zomepirac, zafirlukast.

Additional general NSAID structures and particular NSAID compounds are disclosed in U.S. Pat. No. 6,297,260, incorporated herein in its entirety by reference (especially in the generic formulas of its claim 1 and the recitation of specific list of NSAIDs contained therein and in claim 3, and thiazulidene NSAIDs disclosed in International Patent Application WO 01/87890, also incorporated herein by reference in its entirety.)

Preferred NSAIDs are acetyl salicylic acid, indomethacin, naproxen, ibuprofen, flurbiprofen, ketoprofen, sulindac, etodolac, ketorolac, suprofen, flunixin, sodium diclofenac, flufenamic acid, theophylline, meclofenamic acid, mefenamic acid and tolmetin.

Specific conjugates of Formula VII that may be used according to the present invention are:

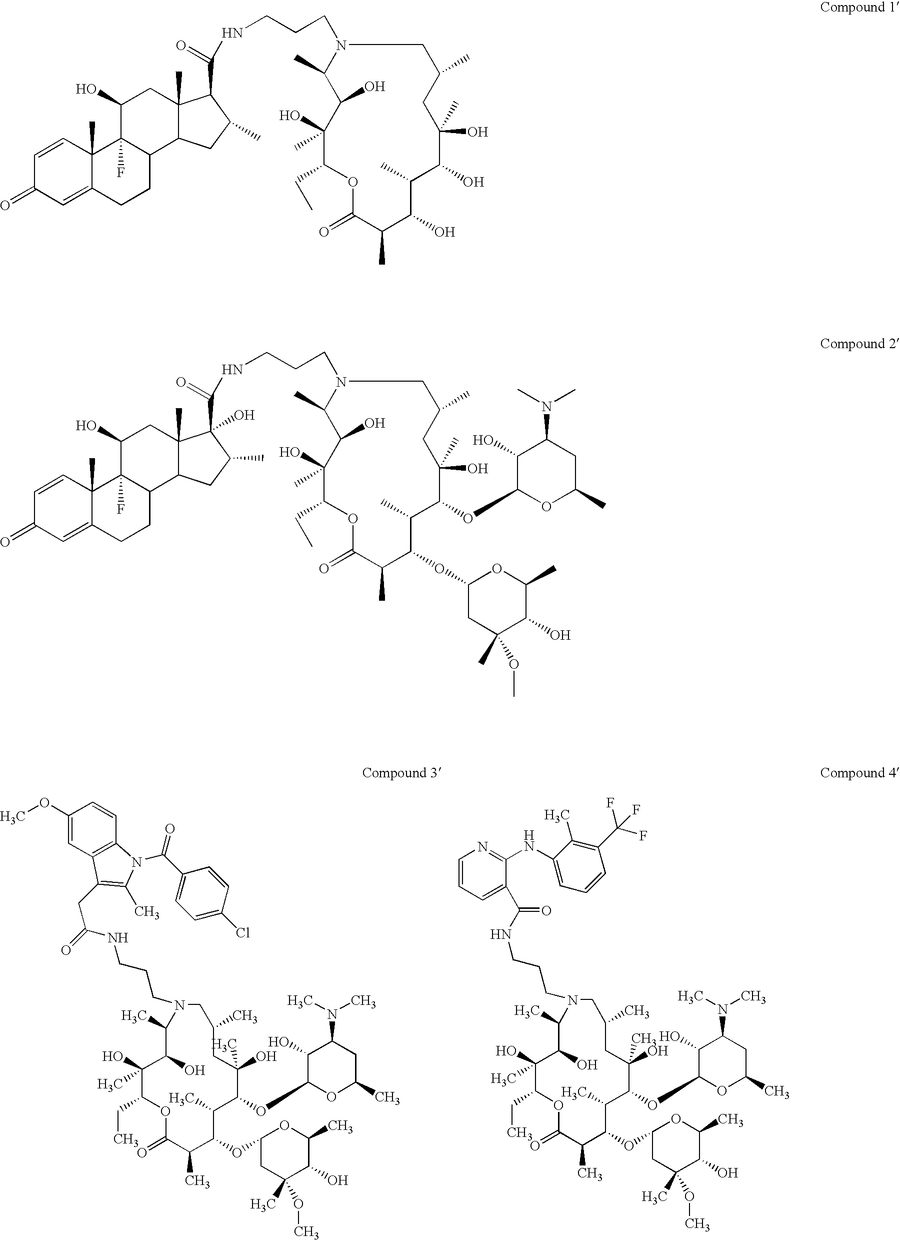

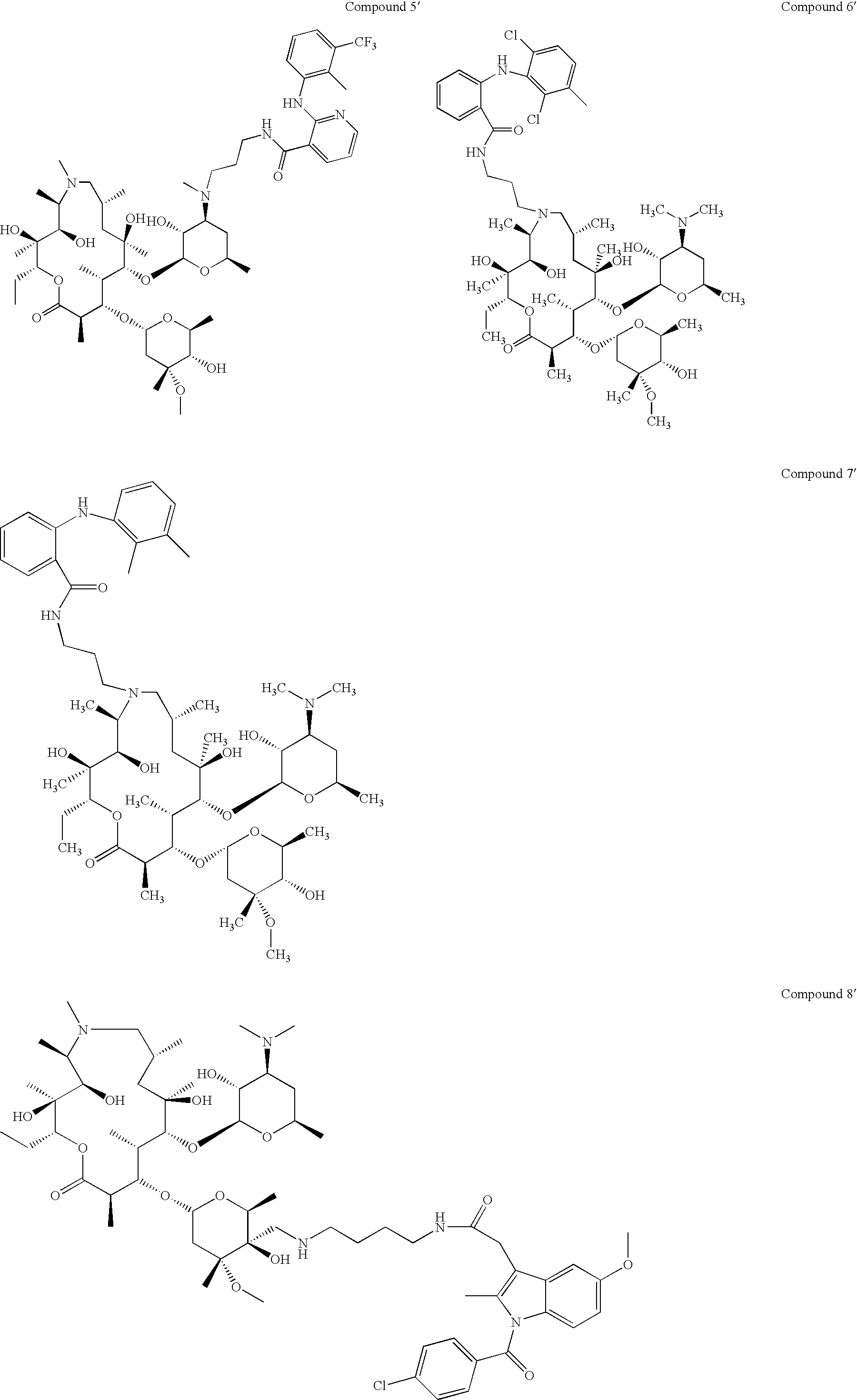

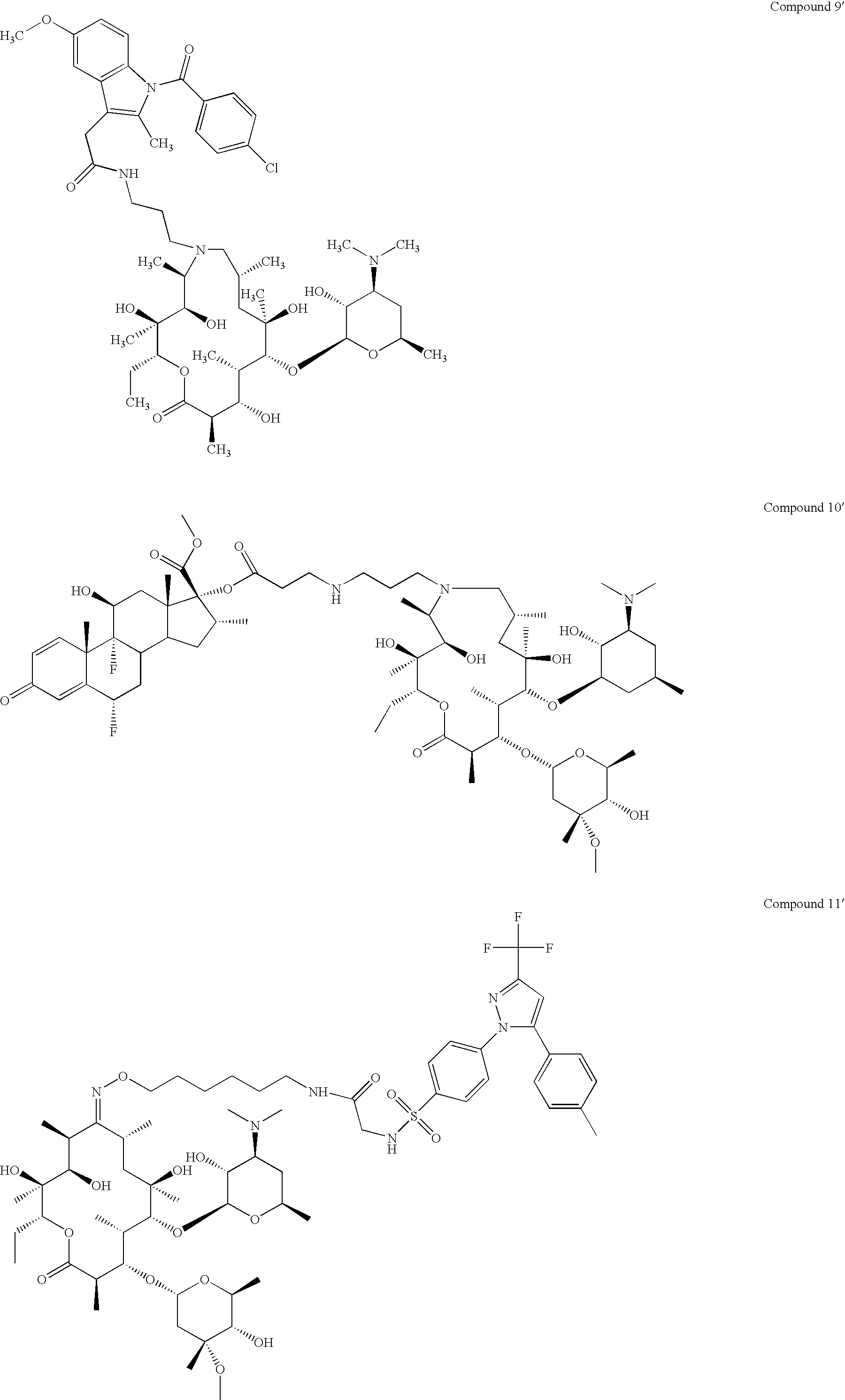

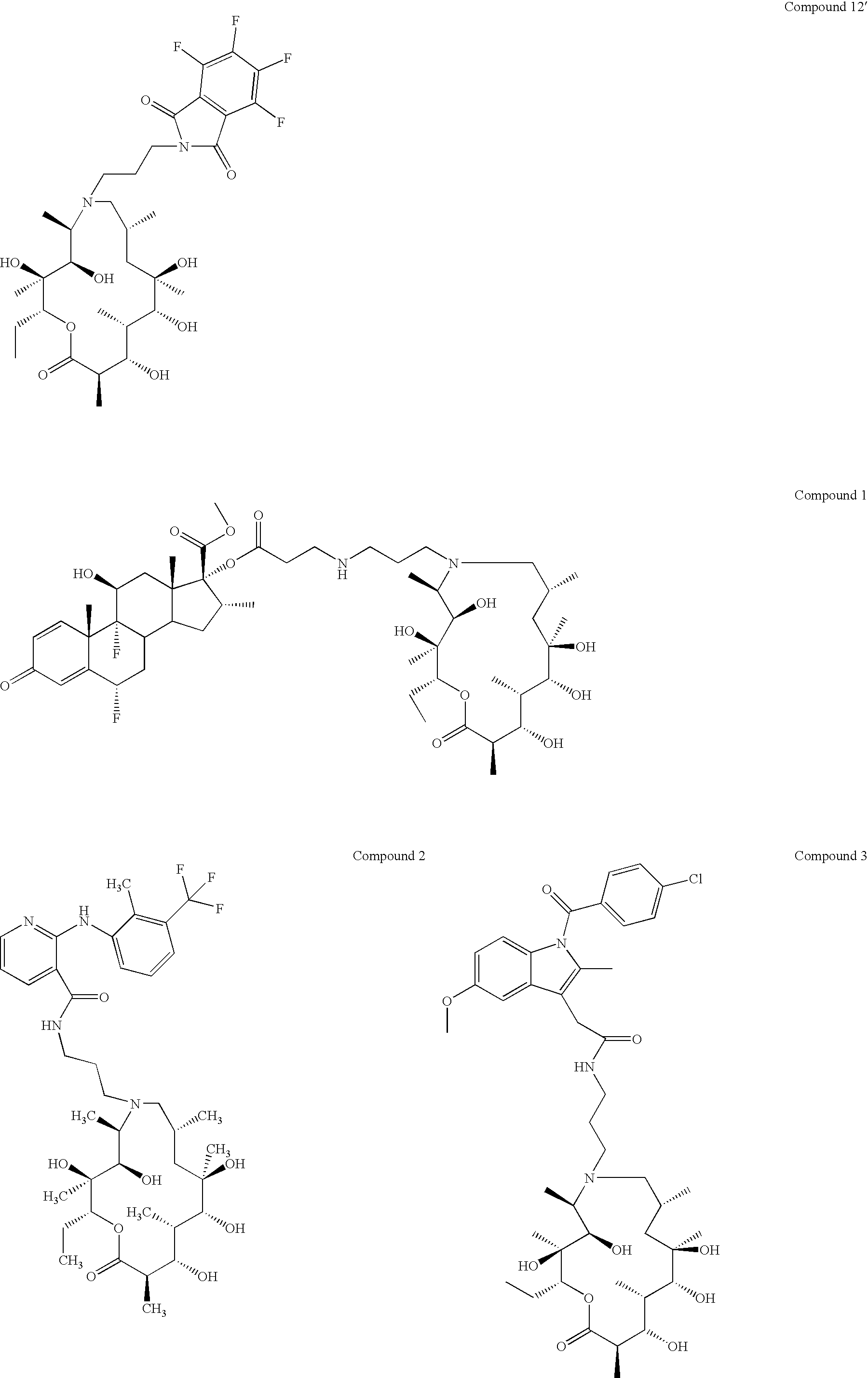

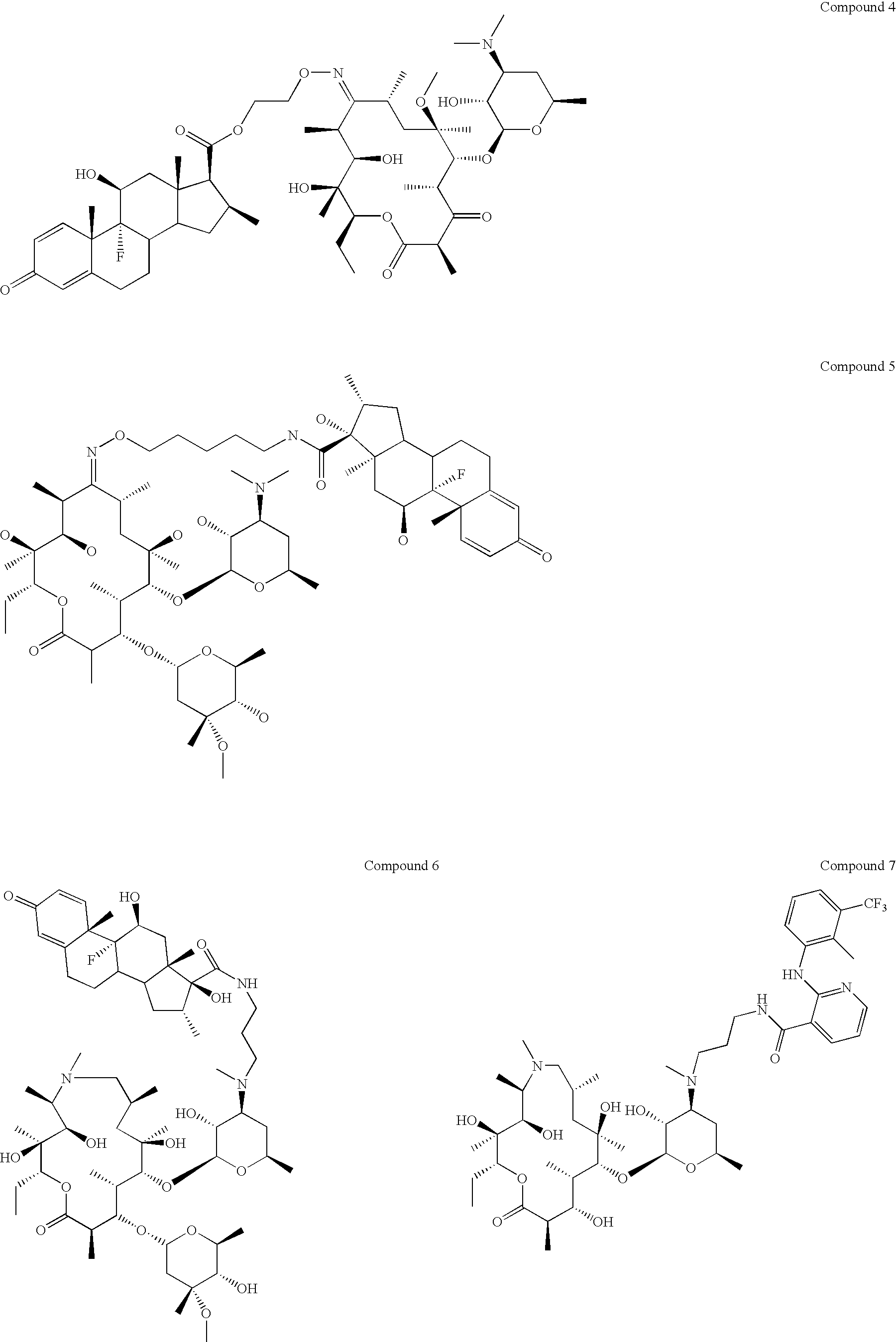

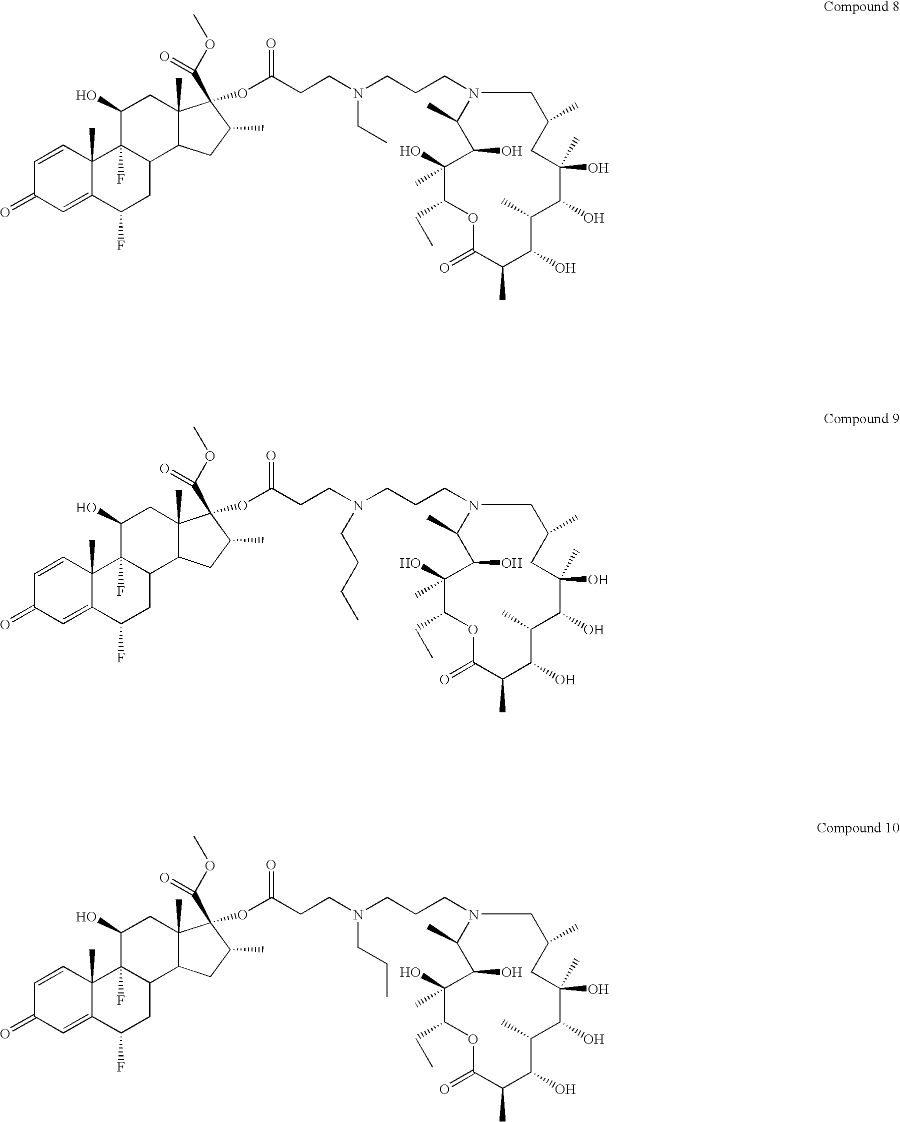

Synthesis of the Conjugates of Formula VII

Generally, the compounds of Formula VII may be obtained in the following way: one end of the chain L is first linked to the macrolide subunit M, and then the other end of the chain is linked to the subunit T; or, one end of the chain L is first linked to the subunit T and then the other end of the chain to the macrolide subunit M, or finally, one moiety of the chain L is linked to the macrolide subunit M, whereas the other moiety of the chain is linked to the subunit T, with the ends of the chain parts being then chemically linked to form the chain L.

It will be appreciated by those skilled in the art that it may be desirable to use protected derivatives of intermediates used in the preparation of the compounds of Formula VII. Protection and deprotection of functional groups may be performed by methods known in the art. Hydroxyl or amino groups may be protected with any hydroxyl or amino protecting group, for example, as described in Green T. W.; Wuts P. G. M. Protective Groups in Organic Synthesis: John Wiley and Sons, New York, 1999. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

Alternatively, conjugate compounds within Formula VII can be prepared by the processes set forth in International Publication Nos. WO 02/055531, WO 04/005310, and WO 04/005309, and in Croatian Patent Application HR 20030324 (and its U.S. counterpart, U.S. Published Application 2005 008003), each of which is incorporated herein by reference in its entirety.

Definitions

The following definitions are set forth to illustrate and define the meaning and scope of the various terms used to describe the present invention.

Bold-faced bonds in formulas contained herein denote bonds raised above the paper level; dash-drawn bonds denote bonds below the paper level, whereas broken lines represent a bond that may be either below or above the paper level. Parallel full and broken lines represent either a single or a double bond. In some instances, a singly-bonded pendent oxygen will be shown; these moieties are hydroxyls as the additional H is assumed to be present to complete the valence. Unless explicitly stated elsewhere herein, the following terms have the meanings ascribed to them below:

“Alkyl” means a linear or branched saturated monovalent hydrocarbon radical chain or branched-chain alkyls include methyl, ethyl, propyl, iso-propyl, butyl, sec-butyl and tert-butyl. Methyl is most preferred. Alkyl groups may be substituted with one up to five substituents including halogen (preferably fluorine or chlorine), hydroxy, alkoxy (preferably methoxy or ethoxy), acyl, acylamino cyano, amino, N—(C1-C4)alkylamino (preferably N-methylamino or N-ethylamino), N,N-di(C1-C4-alkyl)amino (preferably dimethylamino or diethylamino), aryl (preferably phenyl) or heteroaryl, thiocarbonylamino, acyloxy, amino, amidino, alkyl amidino, thioamidino, aminoacyl, aminocarbonylamino, aminothiocarbonylamino, aminocarbonyloxy, aryl, heteroaryl, aryloxy, aryloxyaryl, nitro, carboxyl, carboxylalkyl, carboxyl-substituted alkyl, carboxyl-cycloalkyl, carboxyl-substituted cycloalkyl, carboxylaryl, carboxyl-substituted aryl, carboxylheteroaryl, carboxyl-substituted heteroaryl, carboxylheterocyclic, carboxyl-substituted heterocyclic, cycloalkyl, cycloalkoxy, heteroaryloxy, heterocyclyloxy, and oxycarbonylamino. Such substituted alkyl groups are within the present definition of “alkyl.” The present definition of alkyl carries over to other groups having an alkyl moiety such as alkoxy.

“Alkenyl” means a linear or branched monovalent hydrocarbon radical of two to ten and preferably two to six carbon atoms which has at least one double carbon-carbon bond. Alkenyl groups may be substituted with the same groups as alkyl and such optionally substituted alkenyl groups are encompassed within the term “alkenyl.” Ethenyl, propenyl, butenyl and cyclohexenyl are preferred.

“Alkynyl” means a linear or branched monovalent hydrocarbon radical, having a straight-chain or a branched-chain of two to ten, and preferably two to six carbon atoms and containing at least one and preferably no more than three triple carbon-carbon bonds. Alkynyl groups can be substituted with the same groups as alkyl, and the substituted groups are within the present definition of alkynyl. Ethynyl, propynyl and butynyl groups are preferred.

“Cycloalkyl” means a cyclic group having 3-8 carbon atoms having a single ring optionally fused to an aryl or heteroaryl group. The cycloalkyl groups can be substituted as specified for “aryl” below, and the substituted cycloalkyl groups are within the present definition of “cycloalkyl”. Preferred cycloalkyls are cyclopentyl and cyclohexyl.

“Aryl” means an unsaturated aromatic carbocyclic group having 6-14 carbon atoms having a single ring such as phenyl or multiple fused rings such as naphthyl. Aryl may optionally be further fused to an aliphatic or aryl group or can be substituted with one or more substituents such as halogen (fluorine, chlorine and/or bromine), hydroxy, C₁-C₇ alkyl, C₁-C₇ alkoxy or aryloxy, C₁-C₇ alkylthio or arylthio, alkylsulfonyl, cyano or primary or nonprimary amino.

“Heteroaryl” means a monocyclic or a bicyclic aromatic hydrocarbon ring having from 2 to 10 carbon atoms and from 1 to 4 heteroatoms, such as O, S or N. The heteroaryl ring may optionally be fused to another heteroaryl, aryl or aliphatic cyclic group. Examples of this type are furan, thiophene, pyrrole, imidazole, indole, pyridine, oxazole, thiazole, pyrrole, pyrazole, tetrazole, pyrimidine, pyrazine and triazine, with furan, pyrrole, pyridine and indole being preferred. The term includes groups that are substituted with the same substituents as specified for aryl above.

“Heterocyclic” means a saturated or unsaturated group having a single or multiple rings and from 1 to 10 carbon atoms and from 1-4 heteroatoms selected from nitrogen, sulphur or oxygen, wherein in a fused ring system the other ring or rings can be aryl or heteroaryl. Heterocyclic groups can be substituted as specified for alkyl groups and the thus substituted heterocyclic groups are within the present definition.

“Halogen” means a halogen atom which may be: flourine, chlorine or bromine (most preferably fluorine or chlorine)

The term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopoeia or other generally recognized pharmacopeias for use in mammals, and more particularly in humans.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. However, since memantine is highly soluble, aqueous solutions are preferred. Suitable pharmaceutical carriers are described in “Remington's Pharmaceutical Sciences” by E. W. Martin, 18th Edition. Particularly preferred for the present invention are carriers suitable for immediate-release, i.e., release of most or all of the active ingredient over a short period of time, such as 60 minutes or less, and make rapid absorption of the drug possible.

The present invention also encompasses prodrugs of the Formula VII compounds, i.e., compounds which release an active parent drug according to Formula (VII) in vivo when administered to a mammalian subject. Prodrugs of a compound of Formula VII are prepared by modifying functional groups present in the compound of Formula VII in such a way that the modifications may be cleaved in vivo to release the parent compound. Prodrugs include compounds of Formula VII wherein a hydroxy, amino, or carboxy group of a Formula VII compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of Formula VII or any other derivative which upon being brought to the physiological pH or through enzyme action is converted to the active parent drug.

The present invention also encompasses solvates of the compounds of Formula VII or their salts. Preferred solvates are hydrates.

The compounds of Formula VII have one or more chirality centers and, depending on the nature of individual substituents, they can also have geometrical isomers. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has a chiral center, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomer respectively). A chiral compound can exist as either an individual enantiomer or as a mixture of enantiomers. A mixture containing equal proportions of the enantiomers is called a “racemic mixture”. The present invention encompasses all individual isomers of compounds of Formula VII. The description or naming of a particular compound in the specification and claims is intended to include both individual enantiomers and mixtures, racemic or otherwise, thereof. Methods for the determination of stereochemistry and the resolution of stereoisomers are well-known in the art.

The present invention also encompasses stereoisomers of the syn-anti type, and mixtures thereof encountered when an oxime or similar group is present. The group of highest Cahn Ingold Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

“Low orally-bioavailable” and “low oral bioavailability” means that the conjugate of Formula VII exhibits a bioavailability after its administration by oral, enteral or other mucosal delivery that results in absorption of less than 10% of the administered drug through the gastric mucosa into the blood or plasma, preferably less than 5%, and most preferably less than 2%.

Oral bioavailability is preferably measured through comparing the bioavailability of the compound after oral administration with the bioavailability after intravenous administration of the drug. Equations used to calculate pharmacokinetic parameters including oral bioavailability are as follows:

Area Under the Curve (AUC)

The area under the curve (AUC) is calculated as:

$${AUC}_{( 0 - t )} = {\sum }_{i = 0}^{n = 1} ( {t}_{i + 1} - ti / 2 ) ( {C}_{i} + {C}_{i + 1} ) ,$$

where n is the number of data points

AUC_{∞}=AUC₍₀₋ₜ₎+Cₙ/λ_{z}, where Cₙ is the last concentration and λ_{z} is the elimination rate constant (estimated from the later portion of the plasma/blood concentration vs. time profiles).

The following parameters are determined after intravenous administration:

CLₛ [ml/min/kg]: The rate of elimination of a drug from the body (systemic clearance or organ clearance) normalized to its concentration in an appropriate reference body fluid such as plasma. This is calculated as:
CLₛ=Dose/AUC_{(0-∞)}

Vd [L/kg]: The apparent volume of distribution at the terminal phase based on drug concentration in plasma. This is calculated as:
Vd=Dose/AUC_{∞}*λₛ, normalized by animal weight

t_{1/2} [h]: The half-life of a drug during the terminal phase of plasma/blood drug concentration-time profile. This is calculated as:
t_{1/2}=0.693*Vd/CLₛ

After Oral Administration:

Cₘₐₓ [ex. μg/ml]: The highest drug plasma concentration observed.

Tₘₐₓ [ex. h]: The time at which Cₘₐₓ is reached

F(%): Bioavailability
F=(AUC p.o._{(0-∞)}/AUC i.v._{(0-∞)}/(D_{i.v.}/D_{p.o.}).
In the absence of i.v. data, the oral bioavailability can be estimated if a compound displays low AUC, as defined above. A compound with an estimated oral bioavailability of less than 10% will have an AUC of less than 0.5 μg*h/mL. None of the compounds possessing bioavailability less then 10%, thus entering the scope of the present invention have AUC values above 0.5 μg*h/mL. Thus, molecules defined by Formula VII, having an AUC below 0.5 μg*h/mL will have a bioavailability of less than 10%. It is noted that a number of molecules (for example, compounds 3′, 4′ and 9′ have AUC values of 2.58, 2.18, and 1.13 respectively) have AUC values above 0.5 μg*h/mL and still exhibit bioavailability less than 10% and are therefore within the scope of the present invention. Therefore, compounds having a measured AUC of 0.5 μg*h/mL or more must still be injected through the i.v. route to determine whether they are within the scope of the claimed invention.

“Treating” or “treatment” of a state, disorder or condition includes:

(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,

(2) inhibiting the state, disorder or condition, i.e., arresting, reducing or delaying the development of the disease or a relapse thereof (in case of maintenance treatment) or at least one clinical or subclinical symptom thereof, or

(3) relieving the disease, i.e., causing regression of the state, disorder or condition or at least one of its clinical or subclinical symptoms.

The benefit to a subject to be treated is either statistically significant or at least perceptible to the patient or to the physician.

There are numerous factors which cannot be easily defined. They include:

type of the disease e.g. ulcerative colitis or Crohn's disease

severity of the disease defined by clinical parameters

pharmacogenetic reasons (e.g., responsiveness to glucocorticoid therapy)

Responsiveness of a subject to a treatment is assessed by whether a selected drug used in the acute phase causes the reduction of one or more clinical signs and symptoms described below.

In the context of the present invention, “preventing” is used with reference to maintenance therapy for the prevention of recurrence of a symptom for the disease or any measure of inflammation of the colon, such a marker for inflammation. For example, prevention can be demonstrated in animals that spontaneously develop IBD (e.g. IL-10 deficient mice, TNF ΔARE or SAMP1/Yit mice) and includes the avoidance or the delay of occurrence of disease in treated animals (compared to untreated controls).

An example of “relieving” a subclinical symptom is the observation in a treated individual of abatement in the number of immune cells that secrete pro inflammatory cytokines or lymphokines or a decrease in the mRNA encoding such lymphokines or cytokines.

“Maintenance therapy” is therapy during a phase of the disease, disorder or condition following the achievement of remission (total or partial) of one or more symptoms of the disease until the next flare-up of the disease. Partial remission is the disappearance or alleviation of one or more of the symptoms normally associated with the disease state. The hallmarks of the acute phase include symptoms like nausea, diarrhea, vomiting, fever, abdominal tenderness, pain, cramps, in some cases anemia and malnutrition signs. Anal fistulas can appear. Stools can be bloody or occult bleeding can occur and be determined on assay. White blood cells are moderately elevated, sedimentation rate is often elevated and can be used to monitor the transition from active to remission phase. Hypokalemia, hypoalbuminemia, and hypocalcemia can occur during acute phase. X-ray examination of abdomen and barium enema are used to find lesions in mucosa and inflamed tissue; CT scans and ultrasound can be used for the same purpose. Maintenance therapy starts in the moment when abnormal symptoms previously determined to be present return to normal values. Acute phase therapy usually lasts from 2 to 6 weeks, depending on the patient, and the therapy used. Length of maintenance treatment comprising administration of the compounds of the present invention during maintenance phase typically lasts from the induction of remission until the appearance of disease flare-up or indefinitely if disease is controlled. Perhaps it could be possible to discontinue the administration of the compounds of the present invention after several years of adequate disease control but signs of disease reappearance should carefully be observed.

“Responder” refers to a patient that has previously responded to a treatment for ulcerative colitis or Crohn's disease involving administration of a particular active agents (or combination of active agents) in particular amount or amounts.

“Patient” refers to mammals, preferably humans or domestic animals, more preferably humans.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

Symptoms and signs of disorders and conditions of gastrointestinal tract such as celiac disease and inflammatory bowel disease (Crohn's disease, and ulcerative colitis) include pain, diarrhea, constipation, rectal bleeding, fever, and joint pain malabsorption, chronic vitamin and nutrient deficiency.

Subclinical symptoms include without limitation diagnostic markers for inflammation the appearance of which may precede the manifestation of clinical symptoms. One class of subclinical symptoms is immunological symptoms, such as the invasion or accumulation in an organ or tissue of pro-inflammatory lymphoid cells or the presence locally or peripherally of activated pro-inflammatory lymphoid cells and/or presenting a pro-inflammatory lymphokine or cytokine profile or liberating other mediators of inflammation. Although follow-up of these parameters is not a part of routine clinical practice, there are reports that exhaled nitric oxide (Koek G. H. et al., Respir. Med., 2002, 96:530-5) and that gut inflammation induced increase in permeability could be detected by measuring urinary excretion of lactulose and rhamnose predicts relapse in inactive Crohn disease (Arnott I. D. et al., Scan. J. Gastroentero., 2000, 35:1163-9). Similarly, fecal alpha 1-antitrypsin excretion is also used for assessment of inflammatory bowel diseases (Becker K. et al., Eur. J. Med. Res., 1998, 3:65-70).

Pharmaceutical Compositions

It will be appreciated that pharmaceutical compositions for use in accordance with the present invention may be in the form of orally or enterally administered (or other mucosally administered) suspensions, capsules or tablets, which may be formulated in conventional manner using one or more pharmaceutically acceptable carriers or excipients.

The most preferred oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a lesion or inflammation site has been identified. Or a delayed release can be achieved by a coating that is slow to disintegrate. Or the two (delayed and position release) profiles can be combined in a single formulation by choice of one or more appropriate coatings. Such formulations constitute a further feature of the present invention.

Suitable compositions for delayed release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefosse Deutschland GmbH, D-Weil am Rhein, Germany) and other Suppocire type excipients obtained by interesterification of hydrogenated palm oil and palm kernel oil (C8-C18 triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6,5 to 8. The use of preservatives, masking agents is suitable. The average diameter of micronized particles can be between 1-20 micrometers, or can be less then 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

Pharmaceutical compositions can be prepared by mixing a therapeutically effective amount of the active substance with a pharmaceutically acceptable carrier that can have different forms, depending on the way of administration. Pharmaceutical compositions can be prepared by using conventional pharmaceutical excipients and methods of preparation. The forms for oral administration can be capsules, powders or tablets where usual solid vehicles including lactose, starch, glucose, methylcellulose, magnesium stearate, di-calcium phosphate, mannitol may be added, as well as usual liquid oral excipients including, but not limited to, ethanol, glycerol, and water. All excipients may be mixed with disintegrating agents, solvents, granulating agents, moisturizers and binders. When a solid carrier is used for preparation of oral compositions (e.g., starch, sugar, kaolin, binders disintegrating agents) preparation can be in the form of powder, capsules containing granules or coated particles, tablets, hard gelatin capsules, or granules without limitation, and the amount of the solid carrier can vary (between 1 mg to 1 g). Tablets and capsules are the preferred oral composition forms.

Suppositories for rectal or vaginal administration of the drug can be prepared by mixing a compound of the invention with a suitable nonirritating excipient such as cocoa butter and polyethylene glycols that are solid at ordinary temperatures but liquid at the rectal temperature and will therefore melt in the rectum and release the compound.

Pharmaceutical compositions containing compounds of the present invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

A therapeutically effective amount of the compound of the present invention can be determined by methods known in the art. Since the compound of the present invention is more efficiently delivered to the desired place of action (because of low absorption) than the corresponding unconjugated anti-inflammatory drug alone, a lesser amount of the compound on a molar basis than of the unconjugated anti-inflammatory drug can in principle be administered while still achieving the same therapeutic effect. Furthermore, since administration of the compound results in fewer side effects than with the corresponding unconjugated anti-inflammatory drug the amount delivered to the locus in need of anti-inflammatory treatment can be increased. Thus, the table below serves only as a guide. A threshold therapeutically effective amount of the compound, a pharmaceutically acceptable salt thereof, a solvate thereof, or a prodrug thereof is generally equal to or less than a therapeutically effective amount of the steroid or nonsteroidal anti-inflammatory drug or conjugate on a molar basis. Broad and preferred effective amounts of the compound, a pharmaceutically salt thereof, a solvate thereof, or a prodrug thereof are shown in the table below.

| | Amount of Conjugate, Pharmaceutically Acceptable | |
|---|---|---|
| | Salt Thereof, Solvate Thereof, or Prodrug Thereof | |
| | mg/kg body weight/day of | |
| | the NSAID or steroid (had | mg/kg body weight/day |
| | it been administered alone | of the conjugate |
| Broad | from about 0.001 | from about 0.002 |
| | to about 1000 | to about 2000 |
| Most | from about .001 | from about 0.04 |
| Preferred | to about 100 | to about 200 |
| More | from about 0.01 | from about 0.02 |
| Preferred | to about 50 | to about 100 |
| Preferred | from about 0.1 | from about 0.2 |
| | to about 10 | to about 20 |

The dosage range for treatment of inflammatory bowel diseases and celiac disease is about 1 to about 500 mg per day, depending on the condition of the patient and disease severity. For glucocorticoid conjugates disclosed herein the dose per day is 10-120 mg/day per person, once or twice a day, for NSAID conjugates disclosed herein is 50-500 mg once or twice a day in the acute phase of the disease. However, in the maintenance phase dose can be decreased to a fraction, e.g., 50% or lower of the acute phase treatment, and/or the number of applications can be decreased, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The duration of the treatment can range from weeks to months to years as long as benefits persist and/or side-effects are tolerated.

Dosages and administration regimen can be adjusted depending on the age, sex, physical condition of administered as well as the benefit of the conjugate and side effects in the patient or mammalian subject to be treated and the judgment of the physician, as is appreciated by those skilled in the art.

Methods of Preparation of Steroid Conjugates:

The compound of Formula VII where T is a steroid is prepared by a reaction of the steroid subunit of Formula IX and the amino group of the macrolide subunit of the structure VIII whereby an amide bond is prepared. The starting steroid subunits of the structure X may be obtained by the action of a corresponding halogenalkanoylchloride (preferably 3-chlorpropionylchloride) on the steroid subunit of Formula VII wherein R^{d} is an OH group (Phillipps G. et al., J. Med. Chem., 1994, 37, 3717-3729).

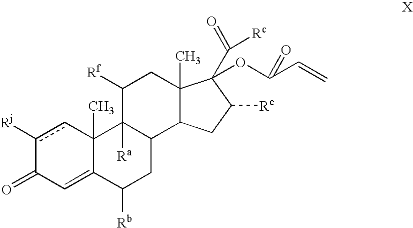

Compounds of Formula VII may generally be obtained in the following way: one end of the linker chain L is first linked to the macrolide subunit M, and then the other end of the chain is joined to the steroid subunit; or, one end of the chain L is first linked to the steroid subunit T and then the other end of the chain to the macrolide subunit M, or finally, one part of the chain is linked to the macrolide subunit M, whereas the other part of the chain is linked to the steroid subunit T. with the ends of the chain parts being then chemically linked to form the chain L.

More general schemes for making the compounds of the invention are apparent to a person of skill in the field of the invention in light of the foregoing. Compounds within Formula VII can be prepared by the following processes.
a) Compounds of Formula VII, where X² in linker L is —NH—, can be formed by reacting (i) a steroid anti-inflammatory subunit represented by Formula XI: 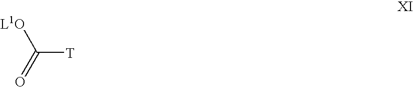
   wherein L¹ represents a leaving group (such as hydroxy), and (ii) a free amino group of a macrolide subunit represented by Formula XII:

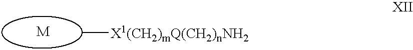

The reaction is generally performed with acid derivatives which have the ability to activate the carboxylic acid group of steroidal anti-inflammatory subunit, such as halogenides, mixed anhydrides and especially carbodiimides (such as -(3-dimethylaminopropyl)-3-ethyl-carbodiimide (EDC)) and benzotriazoles. The reaction proceeds in the presence of a base, such as an organic base (e.g., triethylamine), at room temperature under an inert atmosphere such as nitrogen or argon. The reaction may require several hours to several days to come to completion.

For example, when L is —K—NH— (wherein K is the portion of the L molecule attached to the macrolide) the compound of Formula VII can be formed by derivatizing an NH group on the macrolide ring to an —N—K—(NH₂)— group and reacting the derivatized macrolide with a steroid anti-inflammatory subunit represented by Formula IX:

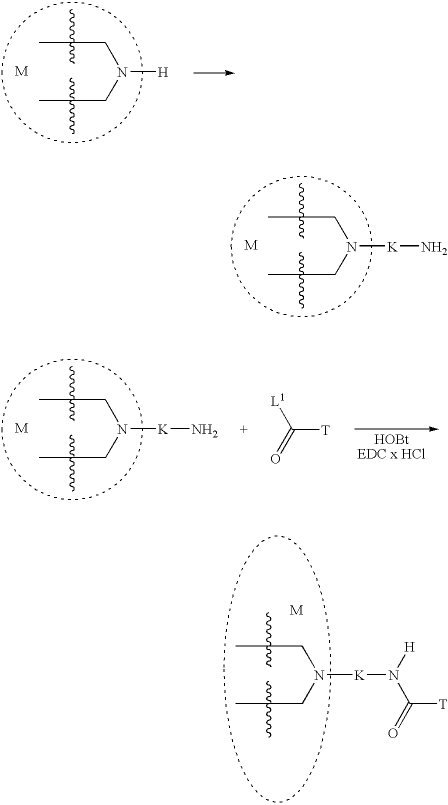

This process may also be performed when the NH group in the macrolide is attached at the 3′ position of a sugar ring S¹ (i.e., a desosamine sugar) of the macrolide:

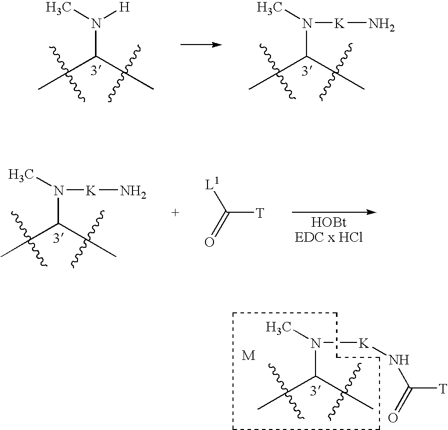

or the 4 position of the sugar ring S²:

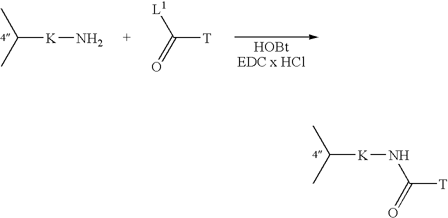

b) Compounds represented by Formula VII, where X² is —OC(═O)—, can be formed by reacting a compound of Formula IX and the free hydroxyl group of a macrolide subunit represented by Formula XIII:

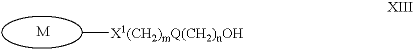

The reaction is generally performed with acid derivatives which have the ability to activate the carboxylic acid group such as halogenides, mixed anhydrides and especially carbodiimides and benzotriazoles. The reaction is typically performed at room temperature under an inert atmosphere such as nitrogen or argon. The reaction may require several hours to several days to come to completion.

The starting macrolide subunits of the structure VIII are known compounds or may be obtained according to the procedures described for analogous compounds, such as those described in Costa A. M. et al., Tetrahedron Letters, 2000, 41:3371-3375, which is hereby incorporated by reference in its entirety. See, also Bright U.S. Pat. No. 4,474,768 and Bright, G. M., et al, J. Antibiot., 1988, 41:1029-1047, both incorporated in their entirety by reference.

For example, when linkage L is —K—O—, the compound of Formula VII can be formed by (1) derivatizing an NH group on a macrolide to an N—K—OH group and (2) reacting the derivatized macrolide with the free carboxylic acid group on a steroid T:

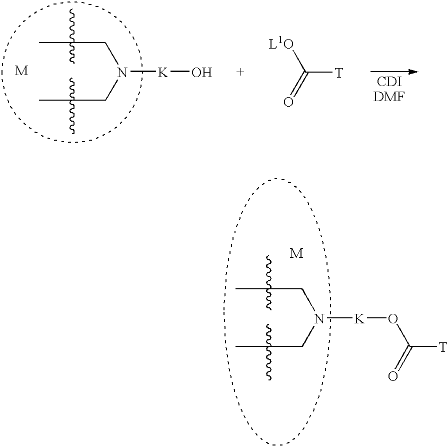

The linkage group —K—OH can be attached to the secondary nitrogen atom of the macrolide subunit as follows. The macrolide subunit is reacted with an alkenoyl derivative, such as CH₂═CH(CH₂)ₘ, C(═O)O-Alkyl (e.g., methylacrylate). The ester group (i.e., —C(═O)O-Alkyl) is then reduced, such as with a metal hydride (e.g., LiAlH₄) in an anhydrous organic solvent, to yield the macrolide subunit having the linkage group —K—OH (i.e., M-K—OH). The reduction is typically performed at a low temperature and preferably at 0° C. or lower.

This process can also be performed when the NH group is attached at the 3′ position of a sugar ring in the macrolide (such as a sugar at the 3 position of the macrolide).

c) Compounds represented by Formula VII, wherein X¹ is —OC(═O)—, Q is —NH— and X² is —NH— can be prepared by reacting a macrolide subunit represented by the formula

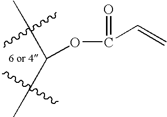

For example this process can be performed when acriloyl group is attached at the C/6 or C/4″ position of the macrolide subunit with steroid subunit represented by Formula:

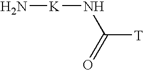

in a solvent, such as acetonitrile, to yield

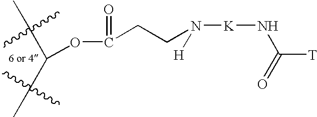

The derivatized steroid (i.e., S—C(═O)—NH—K—NH₂) may be formed by reacting an appropriate amine (having the linkage group —K—NH₂) with a carboxylic acid group or an ester group of a steroid according to Formula IX.

d) Compounds represented by Formula VII, where X² is —NH—, can be prepared by reacting a macrolide subunit and a derivatized steroid subunit having a free amino group as shown below.

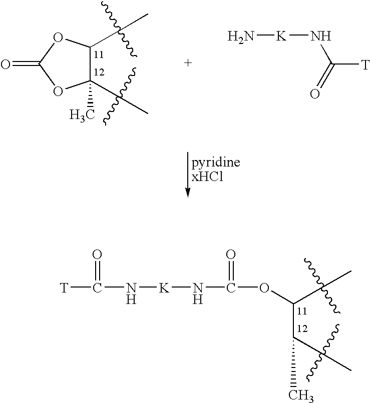

e) Compounds represented by Formula VII, where X² is —NH—, can be prepared by reacting a macrolide subunit and a steroid subunit having a free carboxylic acid group as shown below.

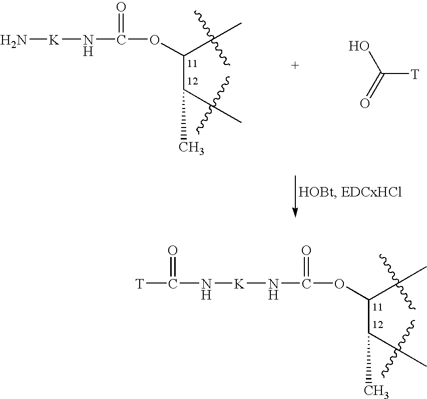

The reactant macrolide subunit can be formed by oxidizing the corresponding macrolide having a hydroxy substituent at the 4″ position on cladinose sugar to obtain a ═O substituent at the 4″ position, converting the 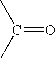
at the 4″ position to an epoxy group 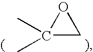
and cleaving the epoxy group with an appropriate reactant(s) to yield the reactant macrolide subunit (M-O—C(═O)—NH—K—NH₂).

f) Compounds of Formula VII can be prepared by reacting a macrolide subunit having a leaving group L² (such as Br), and a steroid as shown below.

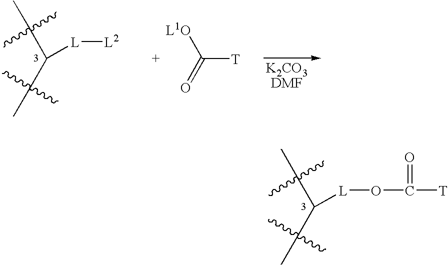

The starting macrolide subunit can be prepared by cleaving the sugar group attached at the 3-position of the macrolide ring and then reacting the macrolide with a reagent of Formula L²-L-L¹, where L² is a leaving group.

g) Compounds of Formula VII can be prepared by reacting a macrolide subunit having a leaving group L² (such as Br), and a steroid as shown below.

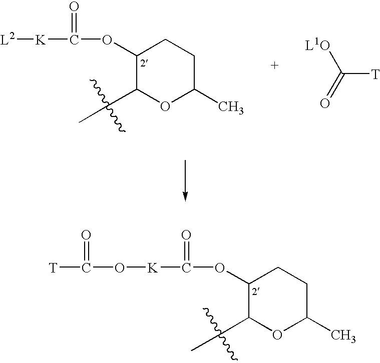

The salts of the compounds represented by Formula VII may be prepared by applying generally known procedures such as, e.g., a reaction of the compounds of the structure VII with a corresponding base or acid in a suitable solvent or mixture of solvents e.g. ethers (diethyl ether) or alcohols (ethanol, propanol or iso-propanol).

The 16-membered ring macrolides are traditionally divided into sub-families based upon the substitution patterns of their aglycones. The principal prototypes of this family can be represented by leucomycin, spiramycin and tylosin.

Tylosin is a representative of 16-membered macrolides, which possesses a highly substituted aglycone with two double bonds (tylonolide) and a third saccharide substituent (β-D-mycinose) in addition to the disaccharide attached to the 5-hydroxyl group. Hydrolysis of mycarose from disaccharide yielded desmycarosyl-tylosin (desmycosin).

Potential sites of modification in desmycosin:

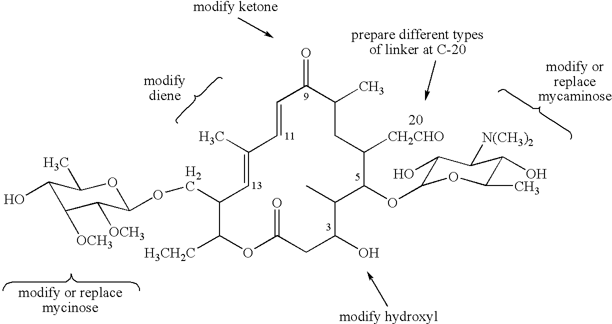

For example, 16-membered ring macrolide hybrid could be prepared by reductive amination of the C-20 aldehyde group.

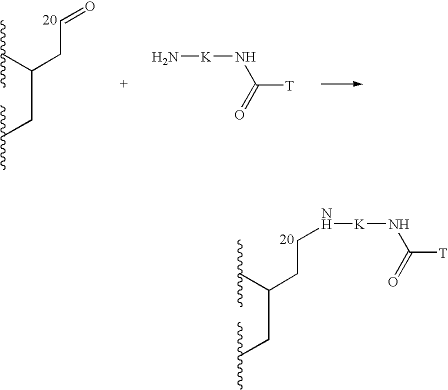

This reaction could be used also for 17-membered azalides like 8a-aza-homodesmycosins and its derivatives (such as di- and tetrahydro derivatives). 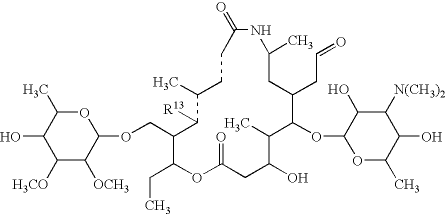
------ represents a single or double bond
   R¹³ is hydrogen or hydroxy

Alternatively, 16-membered ring macrolide derivatisation can proceed by transforming double bonds (e.g., by epoxidation), and cleaving the epoxy group with an appropriate reactant (such as a diamine) to yield the reactant macrolide subunit (M-O—C(═O)—NH—K—NH₂).

Also a ketone in position 9 may be modified by hydroxylamine hydrochloride to yield oxime and then reduced to amine.

If the steroid is an —S(O)ₙ—Rc at position 17, the same linkage schemes can be used.

The steroid subunit may be linked to the macrolide through the 21 hydroxy group in steroids that have such a group. Beginning with a 21-hydroxy steroid cyclic ketal is reacted with an appropriate carboxylic acid halide or an anhydride, preferably in a solvent such as methylene chloride in the presence of a tertiary amine base or pyridine at a reduced temperature (−50 C-300 C). The intermediate so produced is reacted with H₂N-L-M to form compounds of Formula VII

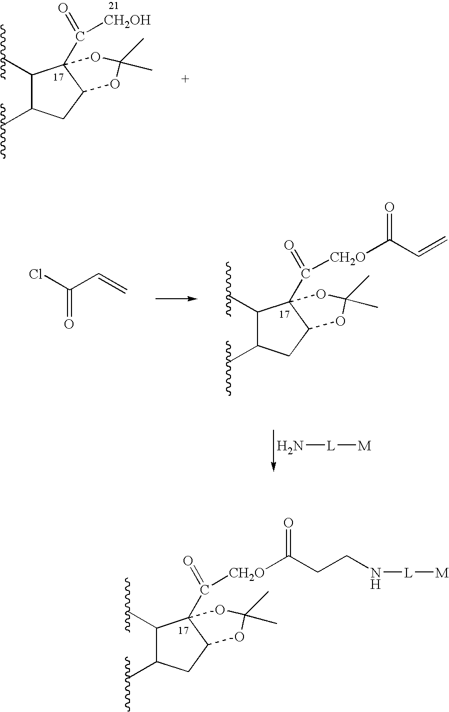

The steroid subunit may also be linked to the macrolide through the 17 position on the steroid subunit. One method for preparing such a compound is as follows:

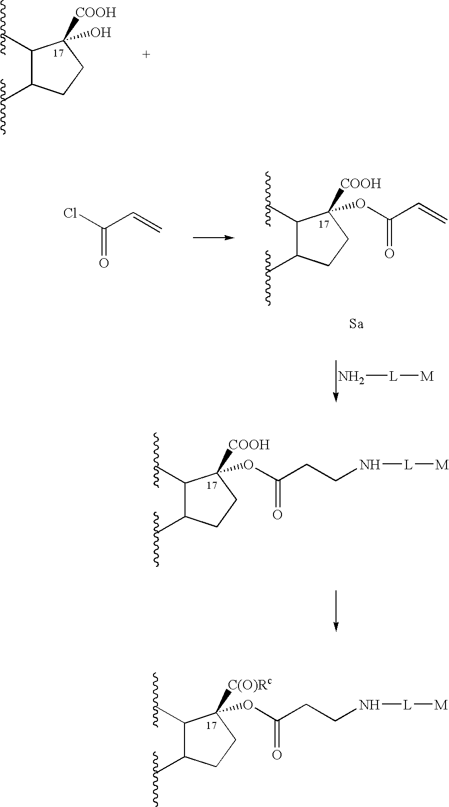

For example, when L is —K—NH— (wherein K is the portion of the L molecule attached to the macrolide) the compound of Formula VII can be formed by derivatizing an NH group on the macrolide ring to an —N—K—(NH₂)— group and reacting the derivatized macrolide with a steroid anti-inflammatory subunit represented by Formula Sa:

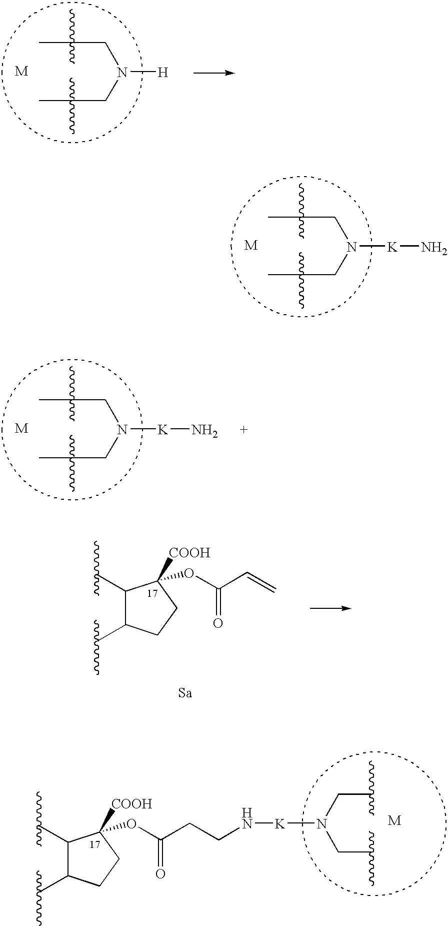

This process may also be performed when the NH group in the macrolide is attached at the 3′ position of a sugar ring S¹ (i.e., a desosamine sugar) of the macrolide:

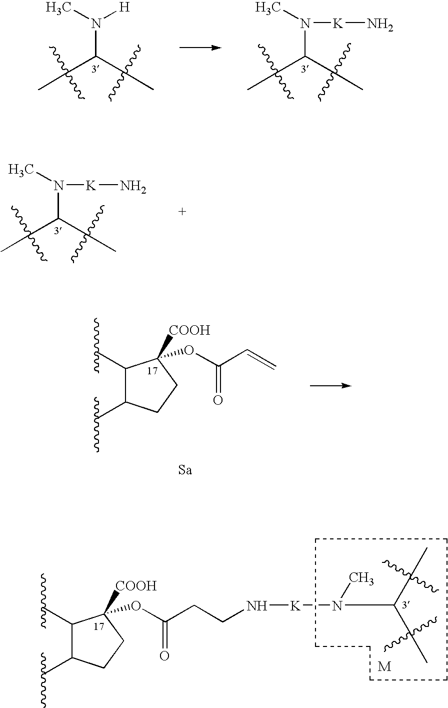

or the 4″ position of the sugar ring S²:

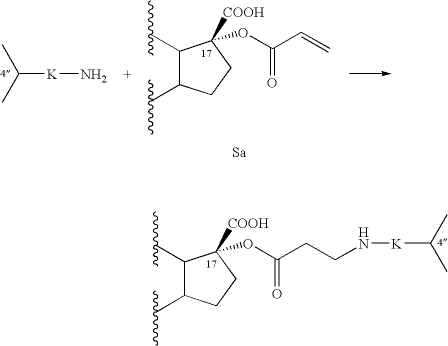

Compounds represented by Formula VII, where X² is —NH—, can be prepared by reacting a macrolide subunit and a steroid subunit having a —C═C— bond as shown below.

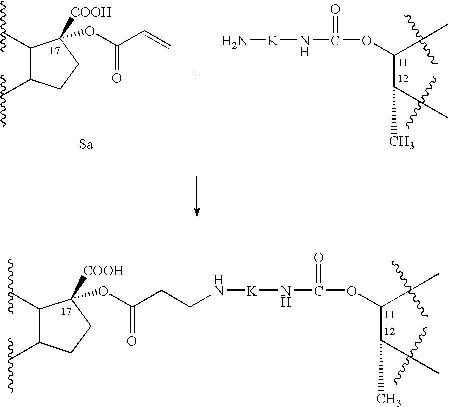

The carboxylic acid group at the 17 position of the starting steroid subunit may be modified prior to the reaction with NH₂-L-M.

The carboxylic acid group at the 17 position of the starting steroid subunit can also be protected prior to the reaction with NH₂-L-M and deprotected after the reaction with NH₂-L-M or the esterification step.

Compounds according to formula VII where T is a nonsteroidal anti-inflammatory subunit may be synthesized by reacting a macrolide intermediate with nonsteroidal anti-inflammatory intermediate following methods known in the art

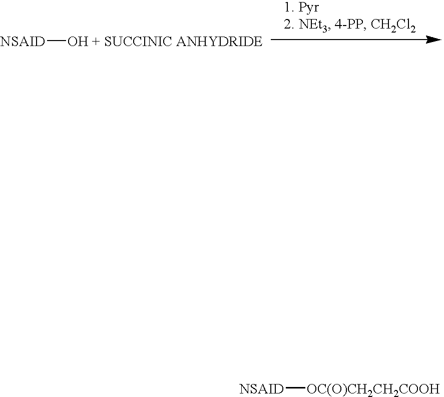

According to Scheme I, NSAID compounds having a hydroxyl group may alternatively be derivatized by the action of succinic anhydride in the presence of pyridine followed by reaction of the intermediate so produced with triethylamine, 4-pyrrolopyridine in methylene chloride to produce NSAID having free carboxylic acid group (Huang C. M. et al. Chem. & Biol. 2000, 7, 453-461, Hess S. et al. Bioorg. & Med. Chem. 2001, 9, 1279-1291) The NSAID derivatives so produced may be coupled either to a linker macrolide compound such as formula XII or directly to a macrolide.

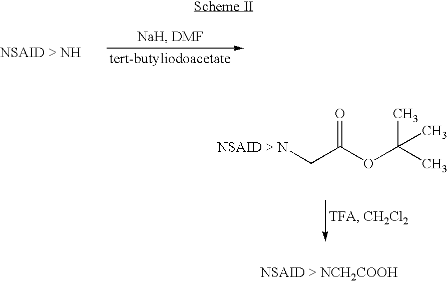

According to Scheme II, NSAID compounds having an amino group may alternatively be derivatized by the action of sodium hydride and tert-butyliodoacetate in N,N-dimethylformamide to produce a (butoxy carbonyl derivative of the NSAID which is then reacted with (trifluoracetic acid in methylene chloride to produce NSAID having free carboxylic acid group (Hess S. et al., Bioorg. & Med. Chem., 2001, 9:1279-91). The NSAID derivatives so produced may be coupled either to a linker macrolide compound such as formula XII or directly to a macrolide.

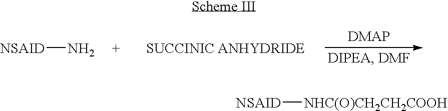

Alternatively by NSAID compounds having an amino group may be derivatized according to Scheme III by the action of succinic anhydride in the presence of dimethylaminopyridine, N,N′-diisopropylethylamine in dimethylformamide to produce NSAID having free carboxylic acid group (Pandori M. W. et al., Chem. & Biol., 2002, 9:567-73). The NSAID derivatives so produced may be coupled either to a linker macrolide compound such as formula XII or directly to a macrolide.

In the case that NSAID compounds contain sulfonamide group, they could be derivatized according to the procedure described in Patent App. US 2003/0055012A1 using ester derivatives of chloro- and bromoacetic acid such as methyl-bromoacetate. Such esters could be hydrolyzed in basic conditions with, for example, LiOH producing free acid which could be coupled either to a linker macrolide compound such as formula XII or directly to a macrolide.

Preparation of the starting macrolide subunits of the structure VIII has been described in PCT/HR02/00001, incorporated by reference in its entirety. See also Bright, U.S. Pat. No. 4,474,768 and Bright G. M. et al., J. Antibiot., 1988, 41:1029-47 each incorporated by reference in its entirety.

For example, when L is —K—NH— (wherein K is the portion of the linking molecule L attached to the macrolide) the compound of Formula VII can be formed by derivatizing an >NH group on the macrolide ring to an >N—K—NH₂ group and reacting the derivatized macrolide with a nonsteroidal anti-inflammatory subunit L¹(C═O)T; wherein L¹ is a leaving group according to Scheme IV. 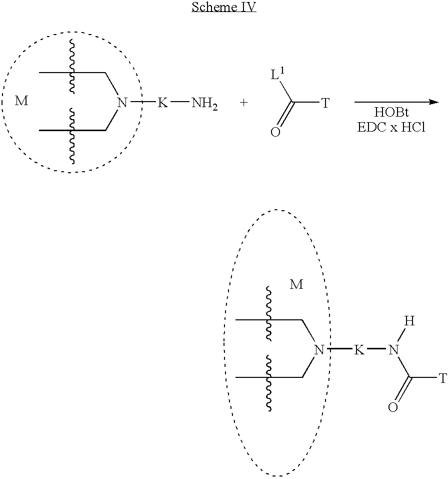
This process may also be performed when the NH group in the macrolide is attached at the 3′ position of a sugar ring S¹ (i.e., a desosamine sugar) of the macrolide according to Scheme V: 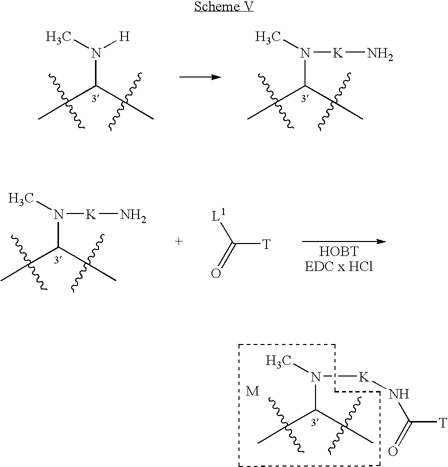
or the 4″ position of the sugar ring S² according to Scheme VI:

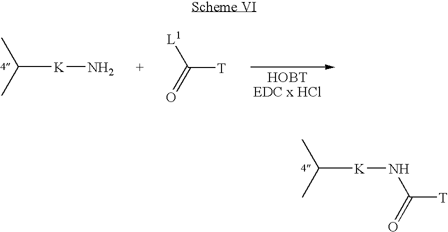

The reactant macrolide subunit can be formed by oxidizing the corresponding macrolide having a hydroxy substituent at the 4″ position on cladinose sugar to obtain a ═O substituent at the 4″ position, converting the 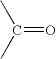
at the 4″ position to an epoxy group 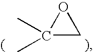
and cleaving the epoxy group with an appropriate reactant(s) to yield the reactant macrolide subunit (M-CH₂—NH—K—NH₂).

b) Compounds represented by Formula VII, where X² is —OC(═O)—, can be formed by reacting a nonsteroidal anti-inflammatory subunit and the free hydroxyl group of a macrolide subunit represented by Formula XIII:

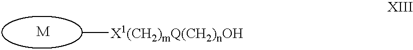

The reaction is generally performed with acid derivatives which have the ability to activate the carboxylic acid group of the nonsteroidal anti-inflammatory subunit, such as halogenides (such as EDC), mixed anhydrides, especially carbodiimides. The reaction is typically performed at room temperature under an inert atmosphere, such as nitrogen or argon. The reaction may require several hours to several days to come to completion.

The starting macrolide subunits of the structure XIII are known compounds or may be obtained according to the procedures described for analogous compounds, such as those described in Costa A. M. et al., Tetrahedron Letters, 2000, 41:3371-75, which is hereby incorporated by reference.

For example, when linkage L is —K—O—, the compound of Formula VII can be formed by (1) derivatizing an >NH group on a macrolide to an >N—K—OH group and (2) reacting the derivatized macrolide with the free carboxylic acid group on a non-steroidal anti-inflammatory subunit according to Scheme VII:

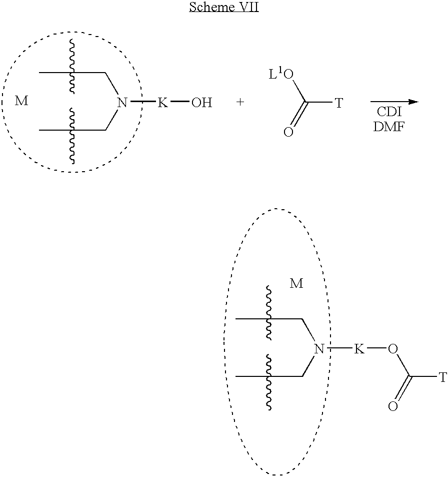

The linkage group —K—OH can be attached to the primary or secondary nitrogen atom of the macrolide subunit as follows. The macrolide subunit is reacted with an alkenoyl derivative, such as CH₂═CH(CH₂)ₘC(═O)O-Alkyl (e.g., methylacrylate). The ester group (i.e., —C(═O)O-Alkyl) is then reduced, such as with a metal hydride (e.g., LiAlH₄) in an anhydrous organic solvent, to yield the macrolide subunit having the linkage group —K—OH (i.e., M-K—OH). The reduction is typically performed at a low temperature and preferably at 0° C. or lower.

This process can also be performed when the NH group is attached at the 3′ position of a sugar ring in the macrolide (such as a sugar at the 5 position of the macrolide).

c) Compounds represented by Formula VII, wherein X¹ is —OC(═O)—, Q is —CH₂— or NH, and X² is —NH—, can be prepared by reacting a macrolide subunit represented by the formula

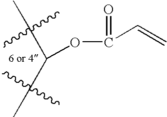

where 4″ is the 4 position on a sugar S², such as a cladinose sugar, and a derivatized nonsteroidal anti-inflammatory subunit having a free amino group represented by the formula:

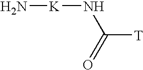

in a solvent, such as acetonitrile, to yield

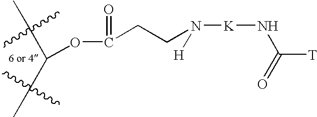

The derivatized nonsteroidal anti-inflammatory subunit (i.e., T-C(═O)—NH—K—NH₂) may be formed by reacting an appropriate amine (having the linkage group —K—NH₂) with a carboxylic acid group of a nonsteroidal anti-inflammatory drug.

d) Compounds represented by Formula VII, where X¹ is —OC(═O)NH— and X² is —NH—, can be prepared by reacting a macrolide subunit and a derivatized nonsteroidal anti-inflammatory subunit having a free amino group as shown below.

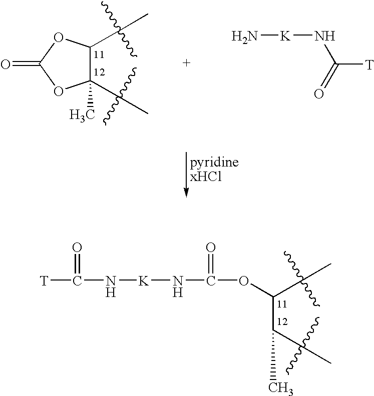

e) Compounds represented by Formula VII, where X¹ is —OC(═O)NH— and X² is —NH—, can be also prepared by reacting a macrolide subunit and a nonsteroidal anti-inflammatory subunit having a free carboxylic acid group as shown below.

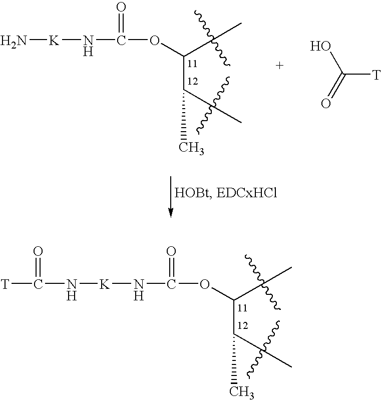

f) The compounds of the Formula VII can be prepared by reacting a macrolide subunit having a leaving group L² (such as Br), and a non-steroidal anti-inflammatory drug as shown below.

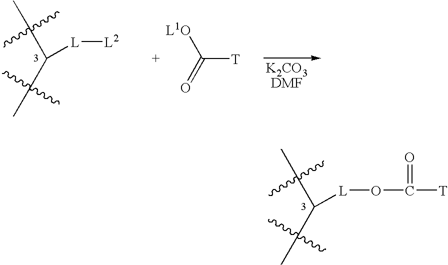

The starting macrolide subunit can be prepared by cleaving the sugar group attached at the 3-position of the macrolide ring and then reacting the macrolide with a reagent of the Formula L²-L-L¹, where L² is a leaving group.

g) The compounds of Formula VII can be prepared by reacting a macrolide subunit having a leaving group L² (such as Br), and a non-steroidal anti-inflammatory drug as shown below.

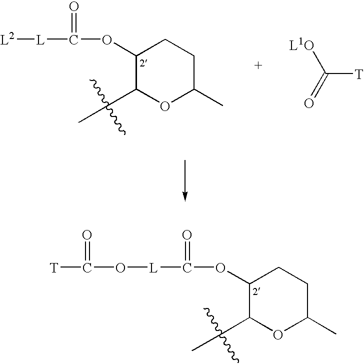

h) Compounds of the Formula VII can be prepared by reacting a macrolide subunit having a leaving group L² (such as Br) and a non-steroidal anti-inflammatory drug as shown below. 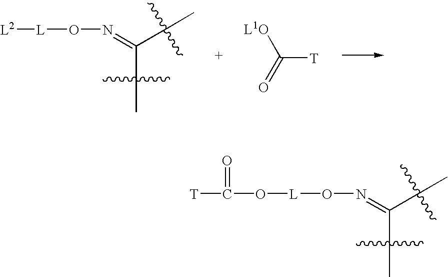
Tablets for Oral Administration

Tablets may be prepared by known art methods, such as direct compression or wet granulation. The active ingredient may be blended with excipients, such as, but not limited to, lactose, croscarmellose sodium and magnesium stearate. The resultant mix may be compressed into tablets using appropriate punches.

Tablets of other strengths may be prepared by altering the ratio of active ingredient to excipient(s) or the compression weight and using punches to suit.

The tablets may be film coated with a film coating suspension using suitable film coating equipment to give a weight of film coat of about 5 to about 10 mg. The release profile of the formulation may be modified by selection of a suitable coating and/or degradable matrix. Alternatively, the release as is known in the formulation art.

### EXAMPLES

The following examples illustrate the invention, but are not limiting.

### Example 1

### Pharmacokinetic Methods for Determining Oral Bioavailability in Rats

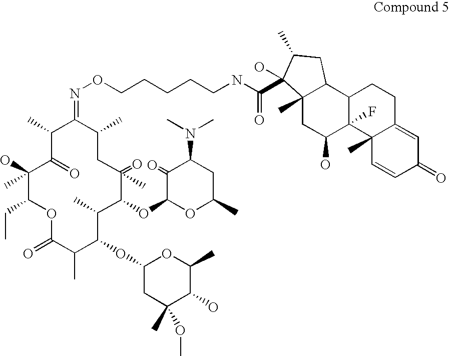

Male Sprague-Dawley rats weighing approximately 200 g were used. After weighing, each rat was given an earmark and placed into a cage. The day before the beginning of the study, animals were randomly distributed into 5 groups with 5 rats/group and placed into solid bottom cages, on wood dust-free bedding (VRF-1 Rodent's diet, Altromin Gmbh). During the experiment, the rats were maintained on a 12-h light-dark cycle. The rats were weighed and fasted for 12 hours prior to p.o. administration.

Compound 5 was administered p.o. in 0.125% carboxymethyl cellulose suspension. The dissolved substance was administered oral at a dose of 30 mg/kg body weight (b.w.), using 2.5 mL syringe (BD) and metal gavage. The administered volume was 10 mL/kg b.w.

Plasma and blood samples were obtained at the following time points after administration: 15, 30, 60, 120, 240, 360 and 480 minutes.

Blood samples were collected by puncturing the lateral tail vein. Prior to each blood sampling, animals were maintained in warming cabinets at 37° C. for 15 minutes. At each sampling time-point 50 μL of blood were collected and added to an Eppendorf test tube (1.5 mL) containing 50 μL demi water. Blood was collected according the time points stated in the protocol. Specimens were stored at −20° C. until analysis.

The concentrations of a compound 5 in the biological matrices were determined by a high performance liquid chromatography method using an acetonitrile and water (acidified by 0.1% formic acid) gradient as the chromatographic medium with tandem mass spectrometry detection (HPLC/MS-MS) (Guidance for Industry, Bioanalytical Method Validation, U.S. Department of Health and Human Services, Food and Drug Administration, May 2001).

Biological samples were prepared by deproteinization with acetonitrile, containing an internal standard. After centrifugation and evaporation of the supernatant fraction, samples were reconstituted in acetonitrile:water with addition of 0.1% formic acid.

HPLC separation was performed on a reverse phase C18 analytical column (Phenomenex C18(2)m 50×2 mm (3 μm) No 182700-5) with a guard column, maintained at ambient temperature, using a gradient of water:acetonitrile [0 min-1 min 10% acetonitrile, 2.5-3.5 minutes 80% acetonitrile, 3.5-5.6 min 10% acetonitrile with addition of 0.1% formic acid at flow rate 0.3 ml/min

MS-MS detection was performed in multi-resolution mode (MRM).

The concentration of the administered compound in biological matrices are calculated by the peak area ratios of compound 5 to an internal standard of roxithromycin using calibration curves and quality control samples. Roxithromycin is also a macrolide antibiotic (synthesized in house). Roxithromycin is commercially available from Sigma-Aldrich Milwaukee Wis.

Non-compartmental analysis was used for pharmacokinetic analysis of the compound in different matrices Jang et al. Med red. Rev. 2001; 21:382-96. The pharmacokinetic parameters were calculated using the PK solutions 2.0 software (Summit Research Services, Pharmacokinetics and Metabolism Software).

The pharmacokinetic parameters were calculated as disclosed herein to obtain the bioavailability wherein F=(AUC p.o._{(0-∞)}/AUC i.v. _{(0-∞)})/(D_{i.v.}/D_{p.o.}).

Amounts of compound 5 detected in plasma and blood were below the limits of detection (LLOQ) and thus no pharmacokinetic parameters could be calculated. These results confirm that oral bioavailability of compound 5 is not measurable, and is anticipated to have low bio-availability when administered to humans.

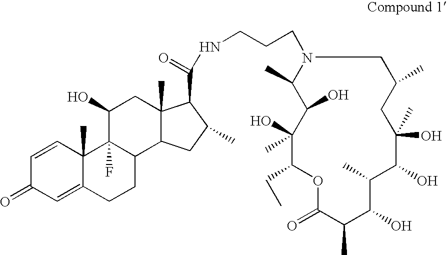

Male Wistar Han rats weighing approximately 220 g were used. After weighing, each rat was given an earmark and placed into a cage. The rats were divided in 3 groups, one with 15 rats for i.v. application, one with 5 rats (p.o.) in order to obtain blood within 24 h, and one with 2 rats in order to collect feces 24 h post administration. During the experiment, the rats were maintained on a 12-h light-dark cycle and allowed free access to food (MUCEDOLA, Italy) and tap water. After grouping, the rats were held in a cage placed in racks. The rats were weighed and fasted for 12 hours prior to p.o. administration.

The compound shown above was administered p.o. in 0.125% carboxymethyl cellulose suspension.

The compound was administered i.v. in the form of its phosphate salt in phosphate buffer saline (PBS).

Plasma and blood samples were obtained at the following time points after administration: 15, 45, 60, 120, 240, 360, 720 and 1440 minutes. Samples were collected by puncturing the tail vein. At each sampling time point 250 μL of blood was collected, from which 50 μL was added to an eppendorf test tube (1.5 mL) containing 100 μL demineralized water. Blood was collected according the time points stated in the protocol up to 24 h post administration. 24 h post administration, after the last collection of blood, the rats were put into a CO₂ atmosphere, and the rat's small and large intestine were removed, cleaned and placed into a Petri dish. Specimens were stored at −20° C. until analyzed.

Amounts of the compound detected in plasma and blood were below the limits of detection (LLOQ) and thus no pharmacokinetic parameters could be calculated. These results confirm that oral bioavailability of the compound is not measurable, and is anticipated to have low bio-availability when administered to humans.

Similarly, the oral bioavailability in rats was determined for the following compounds to provide the following oral bioavailability in rats. These compounds are anticipated to have low bio-availability when administered to humans.

| | | Oral |
|---|---|---|
| | | Bioavailability |
| | Compound | (%) |
| 3′ | 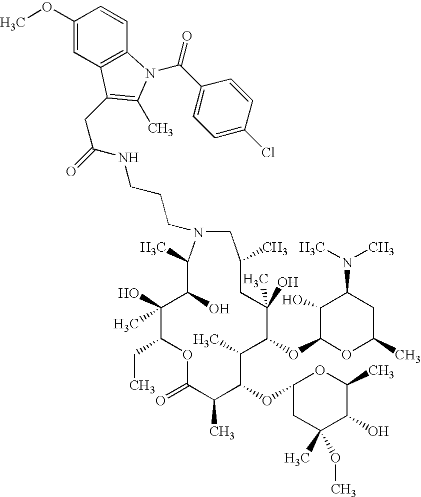 | 3.7% |
| 4′ | 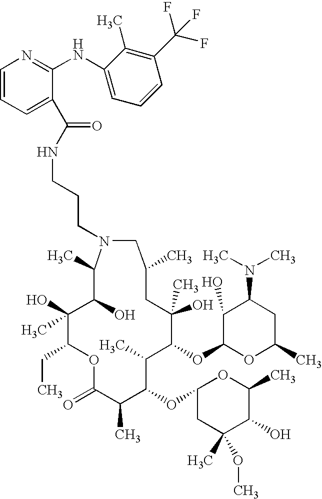 | 6.9% |
| 9′ | 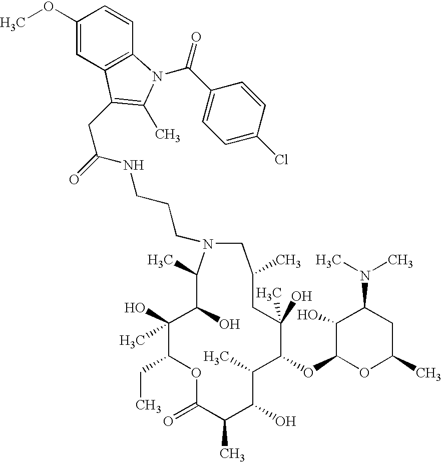 | 2.5% |
| 10′ | 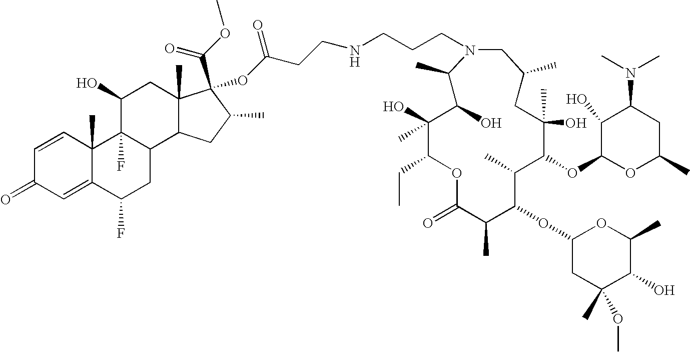 | below limit of quantitation (<1%) |
| 11′ | 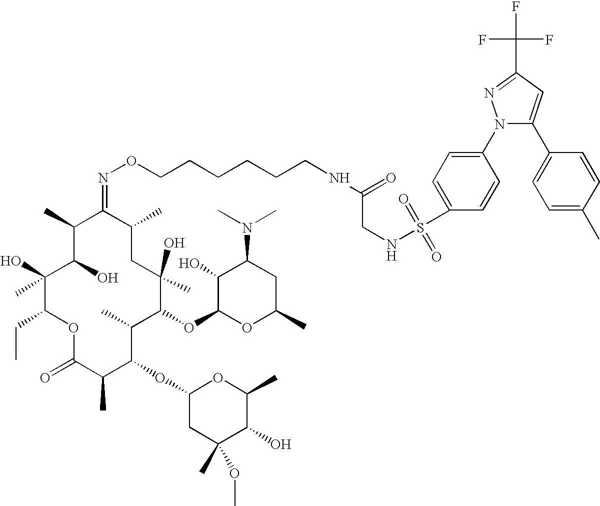 | below limit of quantitation (<1%) |

### Example 2

### Pharmacokinetic Methods for Determining Oral Bioavailability in Mice

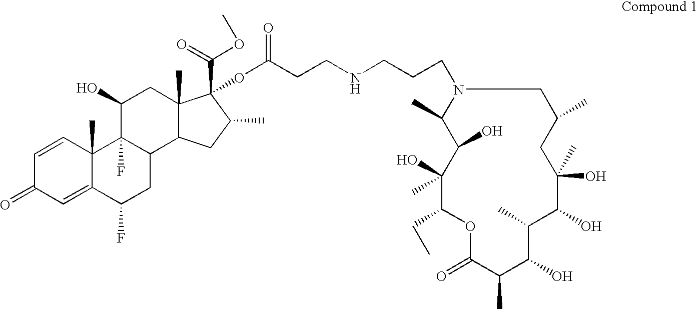

For I.N. and P.O. administration the substance was dissolved in DMSO. 0.5% methyl cellulose was added up to 100% of the total volume. The solution was applied in a volume of 10 ml/kg b.w. (P.O.) and 10 ml/kg b.w. (I.N.). For I.V. administration the substance was dissolved in DMF. PBS was added up to 100% of the total volume. The solution was applied in a volume of 5 ml/kg b.w. (I.V.)

The experiment was carried out on approximately eight week old male BALB/CJ mice (IFFA CREDO, Lyon, France), weighing ˜25 g. Animals were weighed, marked and grouped a day before the beginning of the study. After weighing each mouse was given an earmark and placed into a cage (5 mice per cage). During the experiment mice were maintained on a 12 h light-dark cycle and allowed free access to food (VRF-1 Altromin, Charles River, Hungary) and tap water. Mice were weighed and fasted for 12 hours prior P.O. administration.

The substance was administered P.O. using a 1 mL syringe (BD) and gavage for oral administration. Prior to IV administration, animals were maintained in warming cabinets (SCANBOUR, Denmark) (38° C.) for 15 minutes, in order to facilitate vasodilatation. After formulating the end-use item, substance was administered into the lateral tail vein, using a 1-mL syringe (BD) with a 0.4×19 needle.

Blood was obtained at the following time points after compound administration:

IV (n=10): 10′, 20′, 40′, 60′, 120′, 240′, 360′, 480′, 720′ and 1440 min

PO (n=10): 15′, 30′, 60′, 90′, 120′, 240′, 360′, 480′, 720′, and 1440 min

Prior each blood sampling, mice were anaesthetized using Thiopental®, PLIVA. Whole blood was obtained by preparation and cutting a. carotis comm, and placing it into Vacutainer test tubes with K2EDTA (B-D, lot 9×056). The blood was centrifuged at 3000 rpm within 10 min, after which 100 μl plasma was separated and placed into marked eppendorf test tube 1.5 mL. Blood was collected according the time points stated in the protocol up to 24-h post administration. Specimens were stored at −20° C. until analysis. Soon after obtaining blood, the lungs (after all applications) were extirpated and placed into the previously signed Petri dish. Specimens were stored at −20° C. until analysis. Tissue samples were accurately weighed and homogenized with 4 volumes of water. Thereafter, homogenates were treated as plasma.

HPLC separation was performed on a reverse phase C18 analytical column (Phenomenex C18(2)m 50×2 mm (3 μm) No 182700-5) with a guard column, maintained at ambient temperature, using a gradient of water:acetonitrile [0 min-1 min 5% acetonitrile, 2.5-4.0 minutes 80% acetonitrile, 4.0-6.0 min 5% acetonitrile with addition of 0.1% formic acid at flow rate 0.3 ml/min.

MS-MS detection was performed in multi-resolution mode (MRM).

The concentration of the administered compound in biological matrices are calculated by the peak area ratios of compound 1 to an internal standard of roxithromycin using calibration curves and quality control samples. Roxithromycin is also a macrolide antibiotic (synthesized in house). Roxithromycin is commercially available from Sigma-Aldrich Milwaukee Wis.

Non-compartmental analysis was used for pharmacokinetic analysis in plasma and tissue. The pharmacokinetic parameters were calculated based on average values at each time point using WinNonlin Professional Software, Version 4.1 (Pharsight).

The pharmacokinetic parameters were calculated as disclosed herein to obtain the bioavailability wherein F=(AUC p.o._{(0-∞)}/AUC i.v._{(0-∞)})/(D_{i.v.}/D_{p.o.}).

Concentrations of compound 1 in plasma and lungs after oral administration were below limit of quantitation at all time points, meaning that the compound is not systemically available after oral administration as 0.5% methyl cellulose suspension. These results confirm that oral bioavailability of compound 1 is not measurable, and is anticipated to have low bio-availability when administered to humans.

Similarly, the oral bioavailability in mice was determined for compound 2′ to provide an oral bioavailability of less than 1% (below the limit of quantitation). This compound is anticipated to have a low bio-availability when administered to humans.

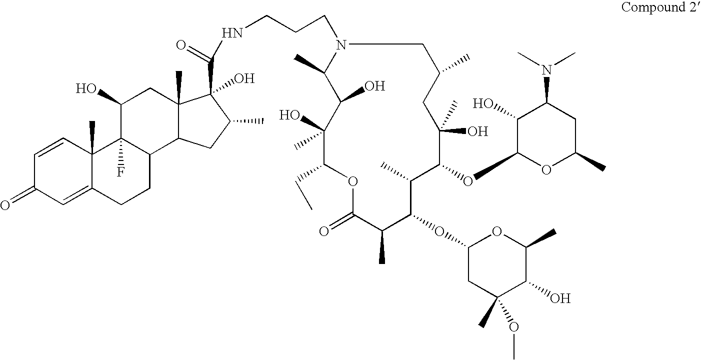

Compounds tested in mice or rats having an AUC of less than 0.5 [symbol] g*h/mL include the compounds in the table below:

| compound | AUC (μg*h/mL) |
|---|---|
| 1 | 0.07 |
| 3 | 0.25 |
| 6 | 0.4 |
| 8 | 0.06 |
| 9 | 0.39 |
| 10 | 0.06 |

These compounds therefore have an oral bioavailability of less than 10%.

### Example 3

### Animal Models for Testing the Effectiveness of Conjugates of Formula VII in the Treatment of IBS and Crohn's Disease

The efficacy of the compounds of Formula VII for the treatment of inflammatory bowel diseases (IBD) can be determined using different in vivo models such as the effect of steroid treatment on the inflammatory parameters of trinitrobenzene sulfonic acid (TNBS)-induced inflammatory bowel disease in rats (Yue G. et al., J. Pharmacol. Exp. Ther., 1996, 276:265-70; Palmen M. J. et al., Dig. Dis. Sci., 1998, 43:2518-25; Nakase H. et al., J. Pharmacol. Exp. Ther., 2001, 297:1122-8; Kankuri E. et al., Inflammation, 2001, 25:301-10), on TNBS-induces IBD in mice (Fiorucci S. et al., Proc. Natl. Acad. Sci., 2002, 99:15770-75), on acetic acid induced acute chemical colitis in rats (Kim Y. S. et al., Arch. Pharm. Res., 1999, 22:354-60), on dextran sulfate sodium (DSS) induced colitis in mice (van Meeteren M. E. et al., Scand. J. Gastroenterol., 2000, 35:517-21, Nakase H. et al., J. Pharmacol. Exp. Ther., 2000, 292:15-21), on disease in SAMP1/Yit mice that spontaneously develop chronic terminal ileitis similar to Crohn's disease (Matsumoto S. et al., Gut, 1998, 43:71-78, Rivera-Nieves J. et al., Gastroenterology, 2003, 124:972-82), on TNF AARE model of Crohn's disease (Kontoyiannis D. et al., Immunity, 1999, 10:387-98, Pizarro T. T. et al., Am. J. Physiol. Gastrointest. Liver Physiol., 2000, 278:G665-9), and colitis in IL-10 deficient mice (Farmer M. A. et al., Proc. Natl. Acad. Sci. USA, 2001, 98:13820-25, Rennick D. M. and M. M. Fort, Am. J. Physiol. Gastrointest. Liver Physiol, 2000, 278:G829-33)

### Example 4

### IL-10-Deficient Mouse Model of Colitis

Mice homozygous for a disrupted interleukin-10 (IL-10) gene support the hypothesis that a disregulated immune response to enteric flora can trigger IBD (Kuhn R. et al., Cell, 1993, 75:263-74). These mice, left untreated invariably develop IBD so this model is particularly suited for testing the preventive effect of compound for development of IBD but can also be used for testing the therapeutic effects on developed disease. The severity of the colitis depends on the inbred strain background in which the disrupted gene is placed (Berg D. J. et al., J. Clin. Invest., 1996, 98:1010-20). The C3H/HeJBir (C3H) strain is a genetic background that is highly susceptible to several experimentally induced forms of IBD. After 10 backcrosses of a disrupted interleukin-10 (IL-10) gene colon lesions are much more severe in C3H mice than in C57BL/6J (B6) mice (Farmer M. A. et al., Proc. Natl. Acad. Sci. USA, 2001, 98:13820-25). Severe lesions of the cecum and colon can be detected in C3H.IL10^{-/-} mice as early as 4 weeks of age, whereas B6 IL10^{-/-} mice develop mild lesions that do not progress in severity. At 6 weeks of age mice are necropsied for tissue collection and analyzed for various phenotypes positively correlated with the progression of colitis. The advantage of this model is that all mice with a disrupted IL-10 gene in C3H genetic background will ultimately develop severe inflammatory bowel disease with the similar intensity. The effectiveness of steroid conjugates (sterolides) is tested by administering them per os from 4 weeks of age at a starting dose of 1-100 mg/kg and following the effect on disease incidence and severitiy. Their activity should be compared to that of standard steroids.

Macroscopic observation and histological evaluation are used to determine the degree of colonic inflammation. The criteria for assessing macroscopic damage and the numerical rating score are as follows: 0, no ulcer, no inflammation; 1, no ulcer, local hyperemia; 2, ulceration without hyperemia; 3, ulceration and inflammation at one site only; 4, two or more sites of ulceration and inflammation; and 5, ulceration extending more than 2 cm. The degree of inflammation on microscopic tissue sections was scored as follows: 0, no leukocyte infiltration; 1, low level of leukocyte infiltration; 2, moderate level of leukocyte infiltration; 3, high vascular density and thickening of the colon wall; and 4 transmural leukocyte infiltration, loss of goblet cells, high vascular density, and thickening of the colon wall.

### Example 5

### Dextran Sulfate Sodium (DSS) Induced Colitis in Mice

Dextran sodium sulfate (2-5% w/v) is given is drinking water to mice. The protocol was as follows:

1. The compounds are dissolved in DMSO (up to a final concentration of 2%) and 0.125% carboxymethyl cellulose and initially applied p.o. at 2 and 10 mg/kg/day for 7 days. The positive control group received only vehicle. DSS solution was provided as drinking fluid for the same time period, and mice were sacrificed on the day 8.

2. DSS solution is administered in drinking water for 7 days. The administration of the compounds started at day 0 and continued to day 14, 21 or 28, and mice were sacrificed on the day 15, 22 or 29.

3. DSS solution is administered in drinking water for 7 days. The administration of the compounds started at day 8 and continued to day 14, 21 or 28, and mice are sacrificed on the day 15, 22 or 29.

Severity of colitis is assessed using a disease activity index (DAI), calculated based on weight loss, stool consistency and bleeding (Cooper H. S. et al., Lab. Invest., 1993, 69:238-49; Hartman G. et al., J. Pharm. Exp. Ther., 2000, 292:22-30). No weight loss is scored as 0 points, weight loss of 1 to 5% as 1 point, of 5 to 10% as 2 points, 10 to 20% as 3 points, and weight loss >20% as 4 points. For stool consistency, 0 point is given for well formed pellets, 2 points for pasty and semiformed stools that do not stick to the anus, and 4 points for liquid stools that do stick to the anus. Bleeding was scored 0 points for no blood in hemoccult, 2 points for positive hemoccult, and 4 points for gross bleeding. The disease activity index is the sum of the combined scores of weight loss, stool consistency and bleeding divided by 3, forming a DAI that ranges from 0.0 (healthy) to 4.0 (maximal activity in colitis). Histological score is assessed in formalin fixed tissue samples of the entire colon. Sections were stained with haematoxylin/eosin and scored by a pathologist unaware of the treatments applied. Infiltration of inflammatory cells, tissue damage and cryp score are subjectively assessed by separate and combined scoring. For infiltration of the inflammatory cells, rare inflammatory cells in lamina propria are scored as 0; increased number of inflammatory cells as 1; confluence of inflammatory cells extending into the submucosa as 2; and transmural extension of the infiltrate as 3. For tissue damage, no mucosal damage is defined as 0, discrete lymphoepithelial lesions are defined as 1, surface mucosal erosion as 2 and extensive mucosal damage as 3. Crypt scoring is performed as follows: 0, intact crypt; 1, loss of bottom one third of the crypt; 2, loss of bottom two thirds of the crypt; 3, loss of entire crypt with the surface epithelium remaining intact; 4, loss of the entire crypt and surface epithelium (erosion). These changes are quantitated as to the percentage involvement by the disease process: 1, 1-25%; 2, 26-50%; 3, 51-75%, and 4, 76-100% of surface area examined. Each piece of tissue was scored with a grade and percent area involvement, and the product of the two was the crypt score.

Clinical improvement in this model is defined by one or more of the following: improvement in DAI, decreased in infiltration of the inflammatory cells, decreased tissue damage and decreased crypt score. These parameters are compared to the values of the positive control group (vehicle treated mice) and animals treated with standard steroids.

### Example 6

### Trinitrobenzene Sulfonic Acid (TNBS)-Induced Inflammatory Bowel Disease in Rats

Colitis is induced in male Wistar rats by intracolonic administration of 10-50 mg of TNBS in 0.25 ml of 50% ethanol. Two days prior to administration of TNBS animals are treated with various doses of sterolide (initially ranging from 0.5 to 10 mg/kg of the compound in 0.125% carboxymethyl cellulose in phosphate buffered saline and 1% DMSO) or vehicle control (0.125% carboxymethyl cellulose in phosphate buffered saline and 1% DMSO) or standard steroid such as for example budesonide (1 mg/kg) for 14 days after the TNBS is applied. Rats were sacrificed, blood samples werewere taken (at day 7 and 15 after application of TNBS) to obtain serum samples followed by macroscopic observation and histological evaluation to determine the degree of colonic inflammation. Macroscopic and histological evaluation of tissue damage was done blindly by pathologist. The criteria for assessing macroscopic damage and the numerical rating score are as follows: 0, no damage; 1, localized hyperemia and/or edema; 2, linear ulcer<half of the width of the colon; 3, linear ulcer>half of the width of the colon; 4, small circular ulcer (<1 cm); 5, circular ulcer between 1 and 2 cm; 6, circular ulcer>2 cm. Scores from individual animals were added together and divided by number of animals to get average microscopic damage. For histological evaluation of colonic injury, each colon is blindly scored according to the following criteria: A) Ulceration: 0, no ulcer, epithelization; 1, small ulcer<3 mm; 2, large ulcer>3 mm; B) Inflammation: 0, no inflammation; 1, mild; 2, moderate; 3, severe; C) Depth of lesion: 0, none; 1, submucosa; 2, muscularis propria; 3, serosa; D) Necrosis: 0, none; 1, mild; 2, severe; E) Granulomas: No, no granulomas; Yes, granulomas present. Maximum score, defined as a sum of all individual scores in this scoring system is 10+granulomas. In some experiments Lekotriene B4 (LTB4) and serum α1 acidic glycoprotein were also measured as an additional indicator of inflammatory activity. Likewise, in selected experiments full thickness specimens from inflamed tissue adjacent to the ulcerated area are excised for measurement of myeloperoxidase (MPO) activity.

Improvement in this experiment is defined by the lower average macroscopic damage and lower histological score, as well as lowering of LTBB4, serum α1 acidic glycoprotein and MPO activity in sterolide treated then in the group of positive control (animals receiving vehicle alone).

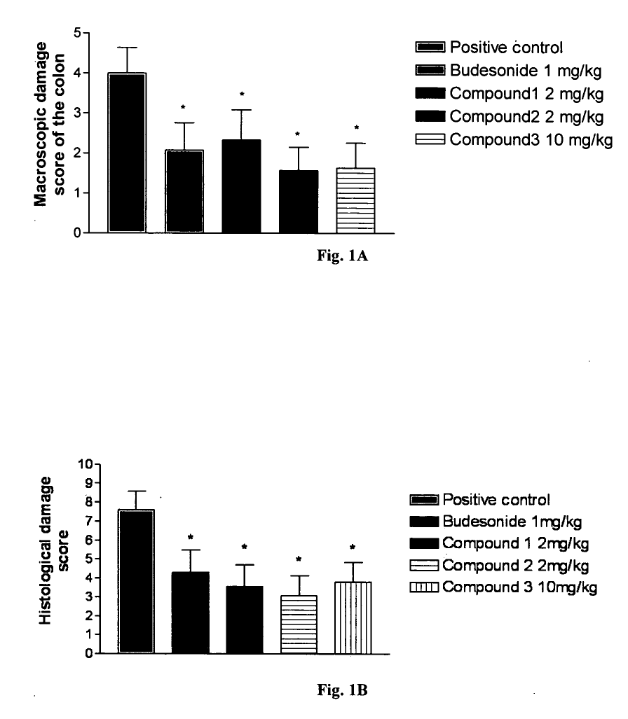

FIG. 1A describes the macroscopic damage score of the colon in the rat model of TNBS induced colitis (Mann-Whitney Test). The macroscopic damage score of the colon is defined as 0—no damage; 1—localized hyperemia and/or edema; 2—linear ulcer<half of the width of the colon; 3—linear>half the width of the colon or small ulcer (<1 cm) 4—circular ulcer<1 cm; 5—circular between 1 and 2 cm; and 6—circular ulcer>2 cm. The compounds 1′, 2′ and 3′ as shown in the table below were compared against budesonide [a known drug useful for the treatment of Crohn's disease in humans] as a treatment control and just a vehicle as a no treatment control. The no treatment control gave a score of 4 while the treatment control, budesonide, at a dosage of 1 mg/kg gave a score of 2. Compounds 1′ (2 mg/kg); 2′ (2 mg/kg) and 3′ (10 mg/kg) all demonstrated comparable or superior efficacy to that of the standard steroid budesonide (1 mg/kg) in the rat model of TNBS induced colitis.

| | Compound |
|---|---|
| 1′ | 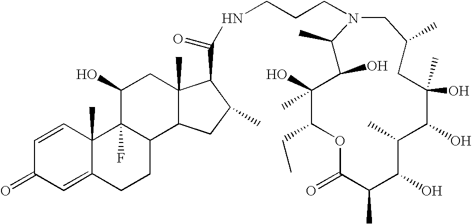 |
| 2′ | 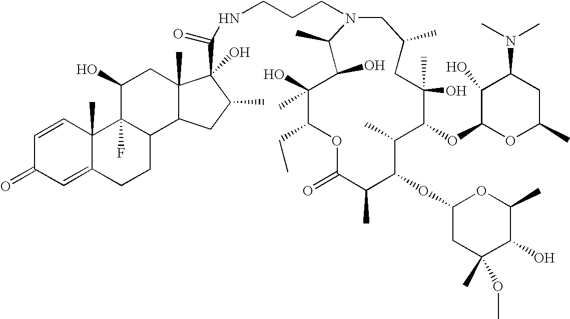 |
| 3′ | 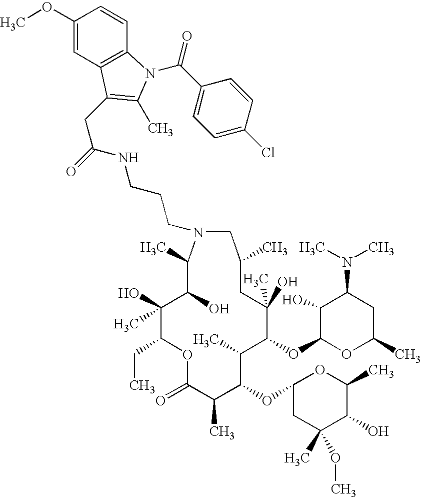 |

FIG. 1B describes effect of conjugates on histological damage score in TNBS induced colitis in rats (Mann-Whitney Test). The histological damage score is defined according to the following criteria: A) Ulceration: 0, no ulcer, epithelization; 1, small ulcer<3 mm; 2, large ulcer>3 mm; B) Inflammation: 0, no inflammation; 1, mild; 2, moderate; 3, severe; C) Depth of lesion: 0, none; 1, submucosa; 2, muscularis propria; 3, serosa; D) Necrosis: 0, none; 1, mild; 2, severe; E) Granulomas: No, no granulomas; Yes, granulomas present. Maximum score, defined as a sum of all individual scores in this scoring system is 10+granulomas. Compounds 1, 2 and 3 were compared against budesonide [a known drug useful for the treatment of Crohn's disease in humans] as a treatment control and just a vehicle as a no treatment control. The no treatment control gave a score of 7,6 while the treatment control, budesonide, at a dosage of 1 mg/kg gave a score of 4,5. Compounds 1′ (2 mg/kg); 2′ (2 mg/kg) and 3′ (10 mg/kg) all demonstrated comparable or superior efficacy to that of the standard steroid budesonide (1 mg/kg) in the rat model of TNBS induced colitis.

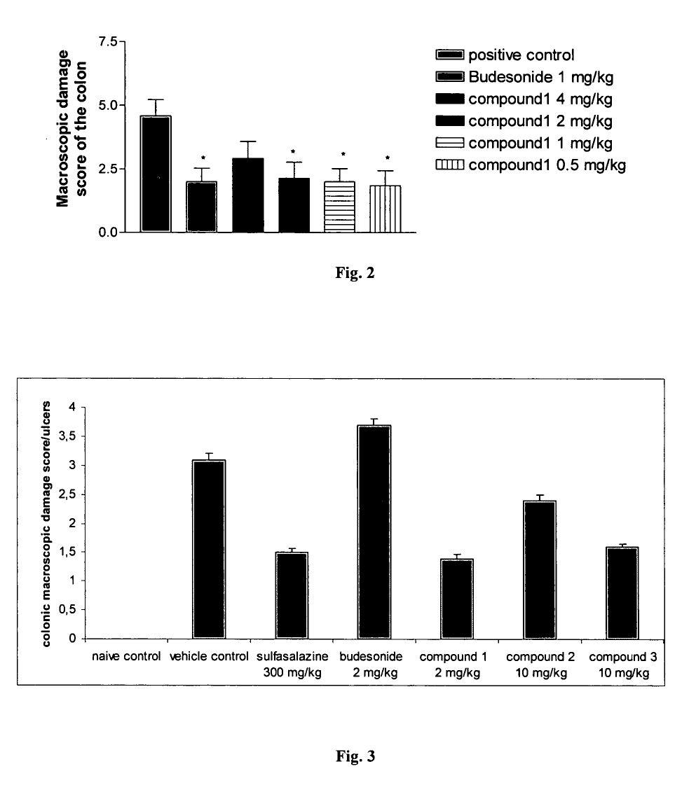

FIG. 2 shows the effect of varying doses of compound 1′ in TNBS induced colitis model in rats (Mann-Whitney Test). The macroscopic damage score is as in FIG. 1A as was the treatment and no treatment controls. Compound 1′ in dosages ranging from 4 mg/kg to 0.5 mg/kg demonstrates comparable or superior efficacy to that of the standard steroid budesonide (1 mg/kg).

### Example 7

### Trinitrobenzene Sulfonic Acid (TNBS)-Induced Inflammatory Bowel Disease in Mice

Effectiveness in Prevention of TNBS-Induced Colitis

TNBS is applied in amounts of 1 to 2.5 mg per mouse to BALB/c and male Swiss Albino mice (6-8 weeks old). Mice are treated with sterolide (1 to 10 mg/kg of the compound in 0.125% carboxymethyl cellulose in phosphate buffered saline and 1% DMSO), vehicle control (0.125% carboxymethyl cellulose in phosphate buffered saline and 1% DMSO) or standard steroid such as budesonide (1-10 mg/kg) or prednisolone (1-10 mg/kg) for 7 days. Mice are monitored for the appearance of diarrhea, loss of body weight, and overall mortality. At the end of experiment, surviving mice are sacrificed and a segment of the colon is excised for macroscopic and microscopic damage evaluation. This animal model is suitable for showing prevention or delay of recurrence or relapse. Damage persists for more then 30 days if untreated, however if treated in the beginning (as described above), no recurrence will occur.

Treatment of Established Colitis

Mice are treated therapeutically with steroids or vehicle control from day 21 to 35 after TNBS administration. Animals are killed 35 days post-TNBS injection.

For histological examination, a sample of colonic tissue located 2 cm above the anal canal is obtained, fixed in 10% buffered formalin phosphate, embedded in paraffin, sectioned, and stained with haematoxylin/eosin. The degree of inflammation on microscopic cross sections is graded semiquantitatively from 0 to 4 as follows: 0, no signs of inflammation; 1, very low level of inflammation; 2 low level of leukocyte infiltration; 3, high level of leukocyte inflammation, high vascular density, and thickening of the colon wall; and 4, transmural infiltrations, loss of goblet cells, high vascular density, and thickening of the colon wall. Improvement in this model is defined by the lower levels of macroscopic damage and inflammation and damage defined by histological score, then in the group of positive control (animals receiving vehicle alone)

### Example 8

### 2,4-Dinitrobenzensulfonic Acid (DNBS)-Induced Distal Colitis in Rats

Group of 10 Wistar male rats weighing 195±5 g were fasted for 24 hours before distal colitis was induced by intra-colonic instillation of DNBS (2,4-dinitrobenzene sulfonic acid, 30 mg in 0.5 mL 30% ethanol/0.9% NaCl) with a catheter of 10 cm in length, followed by gentle injection of air (2 mL) through the catheter to ensure that the solution remain in the colon. Test and standard substances were suspended in 2% of Tween 80 (Sigma, USA). Test substances compound 1, compound 2, compound 3, Budesonide and reference agent sulfasalazine were each administered orally once daily for 7 consecutive days. DNBS was instilled into distal colon of animals at 24 hours after the first dosing and at one hour after the second dosing on day 2. One normal control group was treated without DNBS challenge. The animals were sacrificed 24 hours after the final daily dosing and the colon was removed and weighed. During the experiment, fecal occult blood and stool consistency were monitored daily. Furthermore, when the abdominal cavity was opened before removal of the colon, adhesions between the colon and other organs were noted as was the presence of colonic ulceration after removal and weighing of each colon (a macroscopic damage score). The colon-to-body weight ratio is calculated according to the formula:
(a) Colon-to-Body weight ratio (Colon weight/100 g B. W.) for each rat in respective treatment groups (vehicle blank, vehicle+DNBS, Test substance+DNBS and positive reference agent+DNBS)
   Weight (g) of Dissected Colon×100/Body Weight (g) on 8^{th} day
(b) The Net Increase of Colon weight/100 g B.W.
   For “vehicle+DNBS”: [(vehicle+DNBS)−(vehicle blank)] Mean value of colon-to-body weight ratio
   For “test substance+DNBS”: [(test substance+DNBS)−(vehicle blank)] mean value of colon-to-body weight ratio
(c) The percent decrease of colon weight/100 g B. W.

$$\frac{\begin{matrix}[ ( Vehicle + DNBS ) - ( Test Substance + DNBS ) ] \\ Net Increase of Colon / 100 g B . W .\end{matrix}}{( Vehicle + DNBS ) Net Increase of Colon / 100 g B . W .} \times 100 \%$$
A 30 percent or more (≧30%) reduction in the colon-to-body weight ratio relative to the vehicle-treated group is considered significant anti-inflammatory activity.
   Rat DNBS-induced Abnormal Activity Indices (body weight, diarrhea, rectal bleeding) were determined in all animals daily throughout the study.
(i) Body weight
(ii) Stool consistency (score: 0, normal stools; 1, soft stools; and 2, liquid stools)
(iii) Rectal bleeding (occult fecal blood) (fecal occult blood was measured on a Hemoccult Sensa Card (HemoFEC; Boehringer Mannheim, Barcelona, Spain) score: 0—negative; 1—fecal occult blood test positive; and 2, visible rectal bleeding)

Rat DNBS Histology Readouts at the end of the study Criteria for Macroscopic scoring of damage include the following: (for reference see Mourelle, Gastro, 1996, 110:1093-1097; Appleyard&Wallace, AJP, 1995, 269: G119-125)

| | | Score |
|---|---|---|
| Adhesions | None | 0 |
| | Minimal (colon could be separate easily | 1 |
| | from other tissue) | |
| | Involving several bowel loops | 2 |
| Strictures | None | 0 |
| | Mild | 2 |
| | Severe, proximal dilatation | 3 |
| Ulcers/ | No damage | 0 |
| Inflammation | Focal hyperemia; no ulcers | 1 |
| | 1 site of ulceration/inflammation < 1 cm | 2 |
| | 2 sites of ulceration/inflammation < 1 cm | 3 |
| | Major site(s) of ulceration/inflammation > 1 cm | 4 |
| | If damage > 2 cm increase score by 1 for each | 5+ |
| | additional cm of damage | |
| Wall thickness | <1 mm | 0 |
| | 1-3 mm | 1 |
| | More than 3 mm | 2 |

Total colonic macroscopic damage score: 12+

Test substances were each administered orally once a day for 7 consecutive days and DNBS was instilled intracolonically on the second day. Groups of 10 test animals were sacrificed 24 hours after the final dosing when the colon-to-body weight ratio was recorded. Percent decrease in the colon weight per 100 g body weight relative to the vehicle-treated control was calculated.

As shown in FIG. 3, ccompound 1 and compound 3 caused significant inhibition (≧30%) of the DNBS-induced distal colitis relative to the vehicle group, while compound 2 was associated with a moderate but non-significant anti-inflammatory effect; Budesonide did not show any significant activity.

Sulfasalazine, the positive standard, administered daily and orally at 300 mg/kg for 7 consecutive days showed significant anti-inflammatory activity (44% inhibition).

The results are summarized in the table below:

| | | | % Inhibition Relative |
|---|---|---|---|
| Treatment | Route | Dose | to Vehicle |
| Vehicle | PO | 10 mL/kg × 7 | 0 |
| Budesonide | PO | 2 mg/kg × 7 | 8 |
| compound 1 | PO | 2 mg/kg × 7 | (35) |
| compound 2 | PO | 10 mg/kg × 7 | 27 |
| compound 3 | PO | 10 mg/kg × 7 | (31) |
| Sulfasalazine | PO | 300 mg/kg × 7 | (44) |

A 30 percent or more (≧30%) reduction in the colon-to-body weight ratio relative to the vehicle-treated group is considered significant anti-inflammatory activity (in parenthesis).

### Example 9

### Ulcerative Colitis in the Cottontop Tamarins

The cottontop tamarin is a small new world primate that is unique among animal models of IBD in that it develops a spontaneous form of colitis which shows many similarities to the condition of ulcerative colitis in humans. Animals present clinically with chronic diarrhea and weight loss and may die from the condition if untreated. Both the pathology and response to the therapy with 5-aminosalicylic acid compounds in conjunction with in some cases corticosteroids show close similarities to the condition in humans. A unique feature of the disease in the cottontop tamarin is the development of secondary complications, namely clonic adenocarcinoma and sclerosing cholangtis which are also seen in human ulcerative colitis. Additionally the cottontop tamarin has been used before to evaluate potential therapeutic regimens for treating ulcerative colitis, including Budesonide et al. (Watkins P. E. et al., Gut, 1997, 40:628-33 herein incorporated by reference in its entirety). Therefore, this model has been validated as predictive for the efficacy of drugs in humans.

Cottontop tamarins with clinically manifested ulcerative colitis (diarrhoea and weight loss) and confirmed by endoscopic examination of the colon along with histopathological examination of a rectal biopsy specimen are recruited to this study. Faecal samples are taken from the animals for culture to eliminate known faecal pathogens as cause of these symptoms.

The effectiveness of the conjugates of the invention is tested by administering them p.o. for up to 2 months with doses starting from 1-10 mg/kg.

Standard indicators of disease progression in the cottontop tamarin are used to evaluate response to treatment. These are clinical signs, body weight and histopathological examination of rectal biopsy specimens taken on days 14, 28, 42 and 56. Specimens are graded from 0 (normal) to 5 (severe active disease) on the basis of inflammatory cell infiltrate, crypt architecture and mucosal disruption (Table 1) by a pathologist.

TABLE 1
Histology scoring system

| Acute pathology | Score | Chronic pathology | Score |
|---|---|---|---|
| Polymorphonuclear cells | 1 | Mild chronic | 1 |
| in lamina propria | | inflammation | |
| Polymorphonuclear cells | 2 | Severe chronic | 2 |
| in crypt epithelium | | inflammation | |
| Crypt abscess | 3 | | |
| Crypt destruction | 4 | | |
| Overall maximum score | 5 | | |

Biopsy scores and body weights before and after conjugate treatment are compared using the Friedman non-parametric repeated measures test. Differences are considered significant when p<0.05.

Cottontop tamarins treated with the compounds of the invention showed an improvement of at least 1 based upon the histological scoring system for either acute or chronic pathology in Table 1.

The present invention is not to be limited in scope by the specific embodiments described herein. Various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if fully set forth in this specification.

What is claimed is:

1. A method for the maintenance treatment of an inflammatory disease, disorder or condition of the gastrointestinal tract or the delay or prevention of recurrence of said disease, disorder or condition comprising administering to a human or nonhuman mammalian subject in need thereof an effective amount of a low oral bioavailability conjugate compound of formula VII 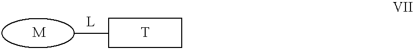
wherein
M is a macrolide subunit selected from the group consisting of 12-, 14-, 15-, 16-, 17-, and 18-membered lactonic ring molecules wherein “membered” refers to the number of carbon atoms or heteroatoms in the lactonic ring said macrolide having the property of accumulating within mammalian immune system cells that mediate inflammatory immune responses;
T is a steroidal or nonsteroidal anti-inflammatory subunit;
L is a linker molecule to which each of M and T are covalently linked,
and pharmaceutically acceptable salts, prodrugs, and solvates thereof in an oral dosage form;
wherein the conjugate of formula VII exhibits less than 10% oral bioavailability.

2. The method according to claim 1, wherein the conjugate of formula VII exhibits less than 5% oral bioavailability.

3. The method according to claim 1, wherein the conjugate of formula VII exhibits less than 2% oral bioavailability.

4. The method of claim 1, where M has the property of accumulating within immune system cells that mediate inflammatory immune responses within the patient.

5. The method according to claim 1, wherein M represents a group of Formula VIII: 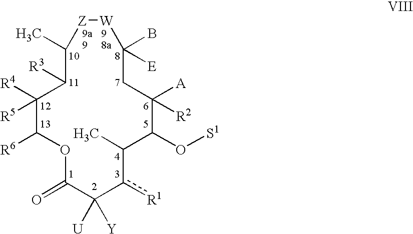
wherein
(i) Z and W independently are 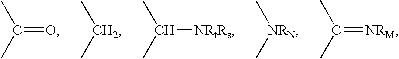
or a bond, wherein
Rₜ and Rₛ independently are hydrogen or alkyl;
R_{M} is hydroxy, alkoxy, substituted alkoxy or OR^{p}
R_{N} is hydrogen, R^{p}, alkyl, alkenyl, alkynyl, alkoxy, alkoxyalkyl, or —C(═X)—NRₜRₛ; wherein X is ═O or ═S;
provided that Z and W cannot both simultaneously be 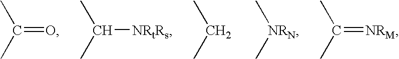
or a bond,
(ii) U and Y are independently hydrogen, halogen, alkyl, or hydroxyalkyl;
(iii) R¹ is hydroxy, OR^{p}, —O—S², or an ═O;
(iv) S¹ is hydrogen or a sugar moiety at position C/5 of the formula: 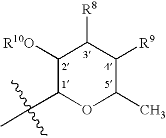
wherein
R⁸ and R⁹ are both hydrogen or together form a bond, or R⁹ is hydrogen and R⁸ is —N(CH₃)R^{y}, wherein
R^{y} is R^{p}, R^{z} or —C(═O)R^{z}, wherein R^{z} is hydrogen or alkyl or alkenyl or alkynyl or cycloalkyl or aryl or heteroaryl or alkyl substituted with C₂-C₇-alkyl, C₂-C₇-alkenyl, C₂-C₇-alkynyl, aryl or heteroaryl;
R¹⁰ is hydrogen or R^{p};
(v) S² is H or a sugar moiety of the formula 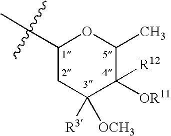
wherein
R^{3′} is hydrogen or methyl;
R¹¹ is hydrogen, R^{p}, or O—R¹¹ is a group that with R¹² and with C/4″ carbon atom forms a >C═O or epoxy group;
R¹² is hydrogen, alkyl, alkyl-R^{p}, R^{p} or a group that with O—R¹¹ group and with C/4″ carbon atom forms a >C═O or epoxy group;
(vi) R² is hydrogen, hydroxy, OR^{p} group, C₁-C₄ alkoxy or substituted alkoxy;
(vii) A is hydrogen or methyl;
(viii) B is methyl or epoxy;
(ix) E is hydrogen or halogen;
(x) R³ is hydroxy, OR^{p}, alkoxy or R³ is a group that with R⁵ and with C/11 and C/12 carbon atoms forms a cyclic carbonate or carbamate, or if W or Z is >N—R_{N}R³ is a group that with W or Z forms a cyclic carbamate;
(xi) R⁴ is C₁-C₄ alkyl;
(xii) R⁵ is hydrogen, hydroxy, OR^{p}, C₁-C₄ alkoxy, or a group that with R³ and with C/11 and C/12 carbon atoms forms a cyclic carbonate or carbamate;
(xiii) R⁶ is hydrogen or C₁-C₄ alkyl;
R^{p} is hydroxyl or amino protective group;
wherein M has a linkage site through which it is linked to the subunit T via the linking group L; provided that the linkage site being at one or more of the following:
a) any reactive hydroxy, nitrogen, or epoxy group located on macrolide ring, sugar moiety S¹, sugar moiety S², or an aglycone oxygen or nitrogen when S¹ and/or S² is cleaved off;
b) a reactive >N—R_{N} or —NRₜRₛ or 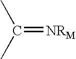
group located on Z or W;
c) a reactive hydroxy group located at any one of R¹, R², R³ and R⁵;
d) any other group that can be first derivatized to a hydroxy or —NRₜRₛ group.

6. The method according to claim 5 wherein S¹ is H and R¹ is OH.

7. The method according to claim 1 wherein L represents a group of Formula IXA or Formula IXB:
X¹—(CH₂)ₘ—X² IXA
X¹—(CH₂)ₘ-Q-(CH₂)ₙ—X² IXB
wherein
X¹ is selected from the group consisting of —CH₂—, —CH₂NH—, —C(═O)—, —OC(═O)—, ═N—O—, —OC(═O)NH— and —C(═O)NH—;
X² is —NH—, —CH₂—, —NHC(═O)—, —C(═O)—, —O— or —OC(═O)—;
Q is —NH— or —CH₂—, wherein each —CH₂— or —NH— group may be optionally substituted by C₁-C₇-alkyl, C₂-C₇-alkenyl, C₂-C₇-alkynyl, C(═O)R^{x}, C(═O)OR^{x}, C(═O)NHR^{x}, wherein R^{x} may be C₁-C₇-alkyl, aryl or heteroaryl;
m and n independently are a whole number from 0 to 8, with the proviso that if Q is NH, n cannot be 0.

8. The method according to claim 1 wherein T represents a steroid subunit of Formula IX: 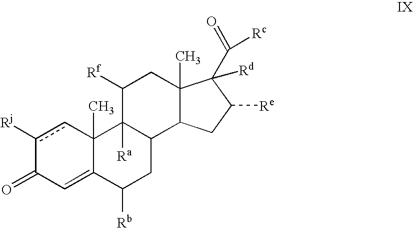
wherein
R^{a}, R^{b}, independently represents hydrogen or halogen;
R^{c} is hydroxy, alkoxy, alkyl, thiocarbamoyl, carbamoyl or a valence-bond attached to X² of chain L;
R^{d} and R^{e} independently represent hydrogen, hydroxy, methyl or C₁-C₄-alkoxy or are each a group that forms a 1,3-dioxolane ring with the other or a valence bond attached to X² of chain L;
R^{f} is hydrogen, hydroxy, chloro, or forms a keto group with the carbon atom to which it is attached; and
R^{j} is hydrogen or halogen.

9. The method according to claims 1 wherein T represents a NSAID subunit selected from the group consisting of:
aceclofenac, acemetacin, acetaminophen, acetaminosalol, acetyl-salicylic acid, acetyl-salicylic-2-amino-4-picoline-acid, 5-aminoacetylsalicylic acid, alclofenac, amino-profen, amfenac, anileridine, azathioprine, bendazac, benoxaprofen, bermoprofen, α-bisabolol, bromfenac, 5-bromosalicylic acid acetate, bromosaligenin, bucloxic acid, butibufen, carprofen, CC 1088, CC 5013, CDC 801, celecoxib, chromoglycate, cinmetacin, cipamfylline, clindanac, clopirac, COX-189, cyclosporine, sodium diclofenac, diflunisal, ditazol, enfenamic acid, etodolac, etofenamate, etoricoxib, felbinac, fenbufen, fenclozic acid, fendosal, fenoprofen, fentiazac, fepradinol, FK-506, flufenamic acid, flunixin, flunoxaprofen, flurbiprofen, glutametacin, glycol salicylate, ibufenac, ibuprofen, ibuproxam, indomethacin, indoprofen, isofezolac, isoxepac, isoxicam, JTE-522, ketoprofen, ketorolac, L-745337, leflunomide, lornoxicam, loxoprofen, meclofenamic acid, mefenamic acid, meloxicam, mesalamine, mesalazine, methotrexate, metiazinic acid, mofezolac, montelukast, mycophenolic acid, naproxen, niflumic acid, olsalazine, oxaceprol, oxaprozin, oxyphenbutazone, parsalmide, perisoxal, phenyl-acethyl-salicylate, phenylbutazone, phenylsalicylate, teophyline, pyrazolac, piroxicam, pirprofen, pranoprofen, protizinic acid, rapamycine, rofecoxib, salacetamide, salicylamide-O-acetyl acid, salicylsulphuric acid, salicin, salicylamide, salsalate, sulindac, sulfasalazine, suprofen, suxibutazone, tenoxicam, thalidomide, tetrafluorthalidomide, tiaprofenic acid, tiaramide, tinoridine, tolfenamic acid, tolmetin, tomoxiprol, tropesin, valdecoxib (Searle), xenbucin, ximoprofen, zaltoprofen, zomepirac, zafirlukast.

10. The method according to claim 1 whereby the compound of the formula VII has the structure:

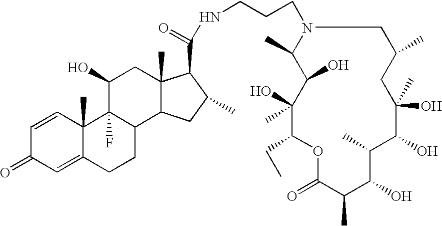

11. The method according to claim 1 whereby compound of the formula VII has the structure:

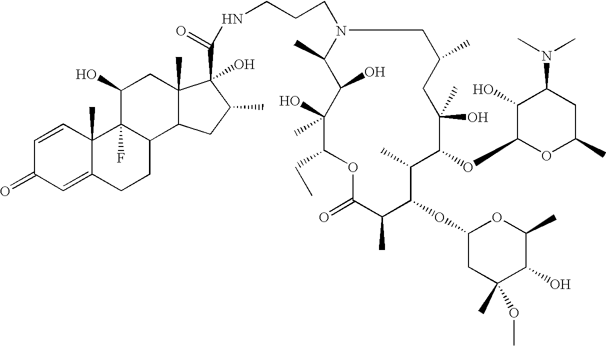

12. The method according to claim 1 whereby compound of the formula VII has the structure:

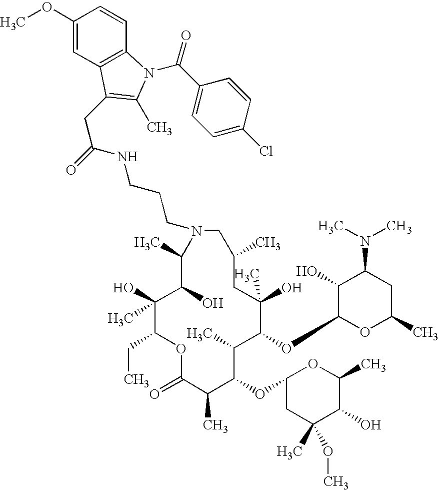

13. The method according to claim 1 whereby compound of the formula VII has the structure:

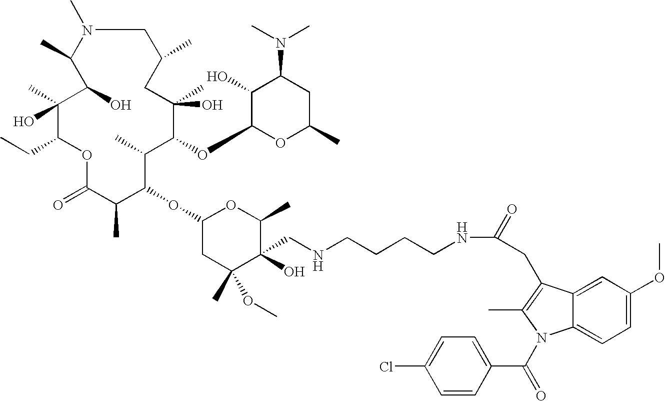

14. The method according to claim 1 whereby compound of the formula VII has the structure:

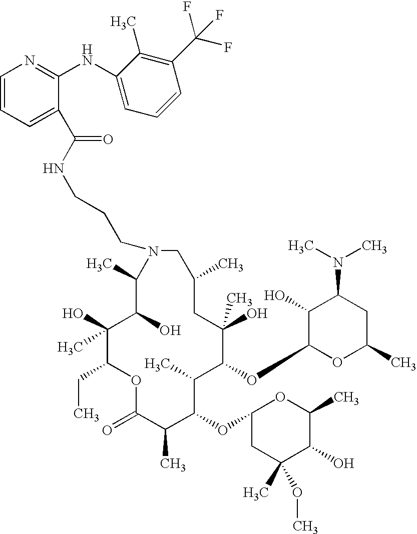

15. The method according to claim 1 whereby compound of the formula VII has the structure:

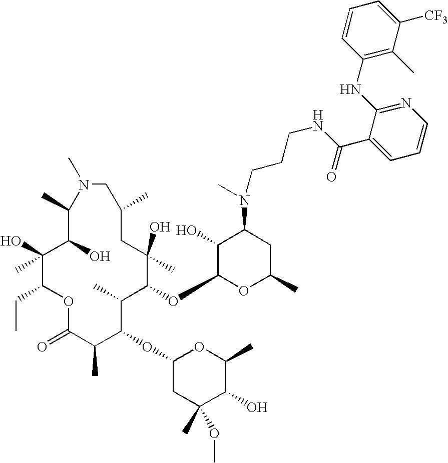

16. The method according to claim 1 whereby compound of the formula VII has the structure:

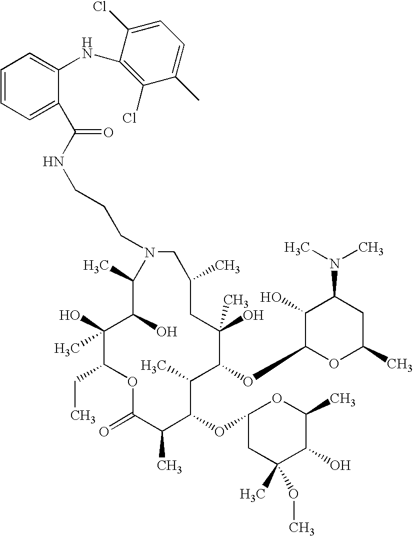

17. The method according to claim 1 whereby compound of the formula VII has the structure:

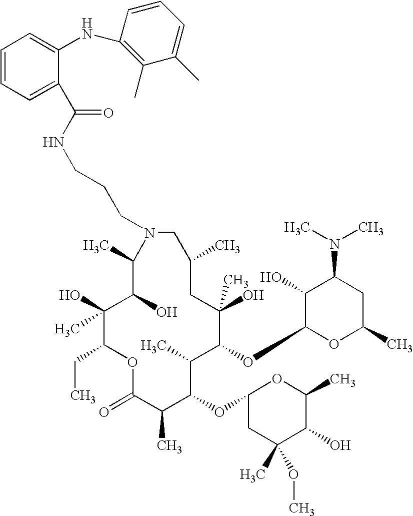

18. The method according to claim 1 whereby the compound of the formula VII has the structure:

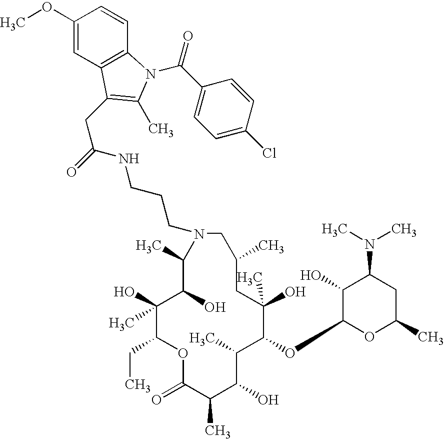

19. The method according to claim 1 whereby the compound of the formula VII has the structure:

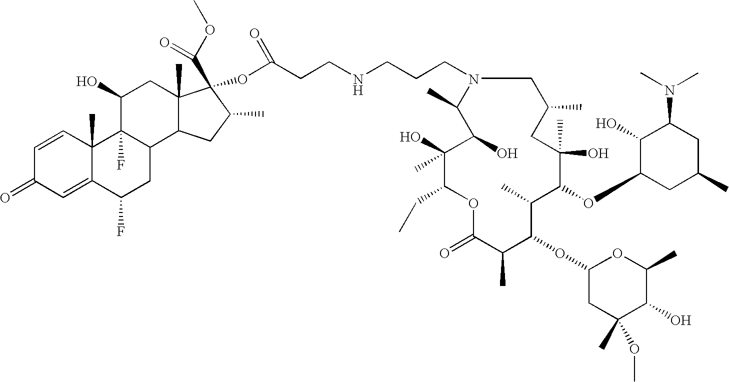

20. The method according to claims 5 whereby the compound of the formula VII has the structure:

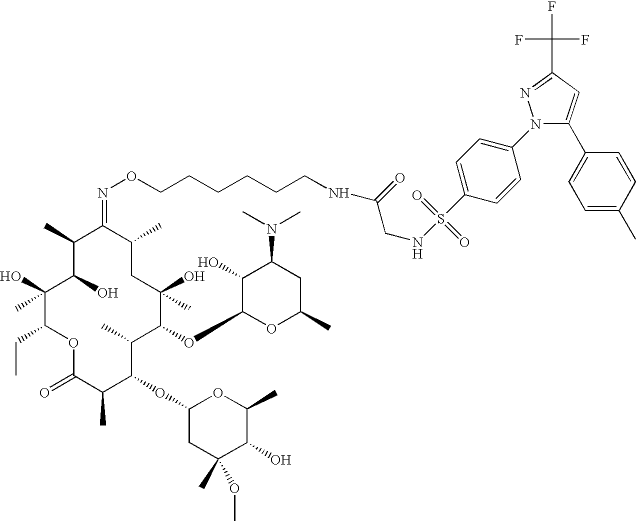

21. The method according to claim 1 whereby the compound of the formula VII has the structure:

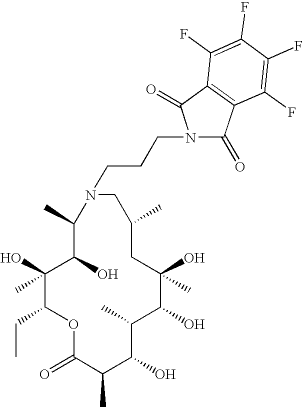

22. The method according to claim 1 whereby the compound of the formula VII has the structure:

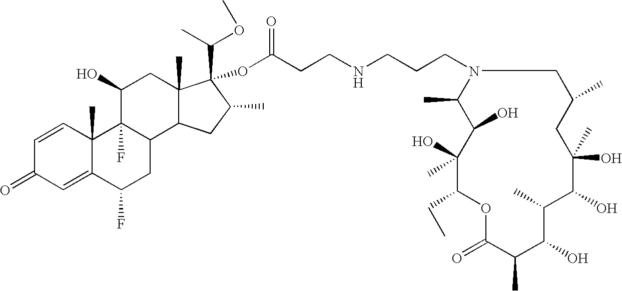

23. The method according to claim 1 whereby compound of the formula VII has the structure:

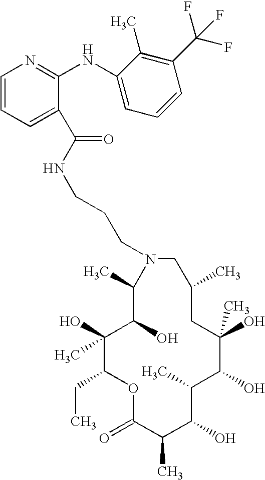

24. The method according to claim 1 whereby compound of the formula VII has the structure:

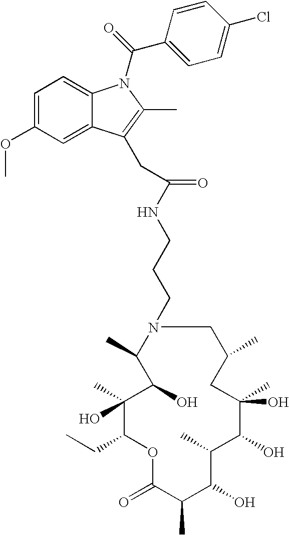

25. The method according to claim 1 whereby compound of the formula VII has the structure:

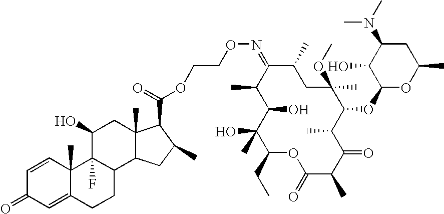

26. The method according to claim 1 whereby compound of the formula VII has the structure:

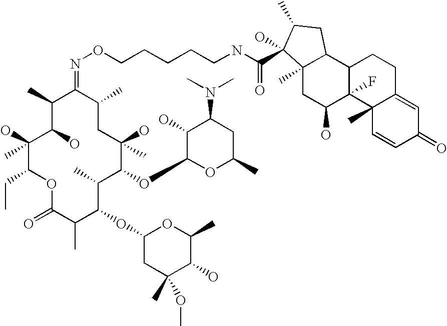

27. The method according to claim 1 whereby compound of the formula VII has the structure:

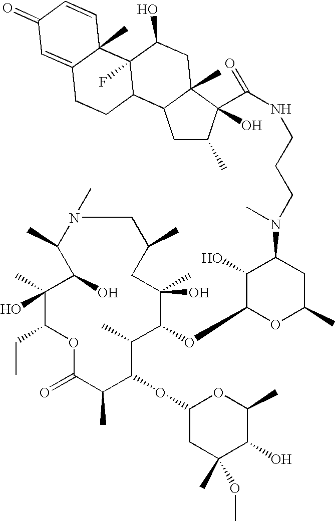

28. The method according to claim 1 whereby compound of the formula VII has the structure:

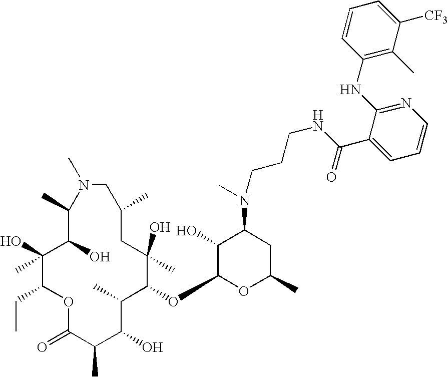

29. The method according to claim 1 whereby compound of the formula VII has the structure:

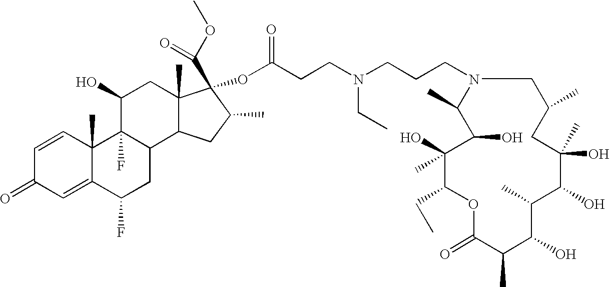

30. The method according to claim 1 whereby the compound of the formula VII has the structure:

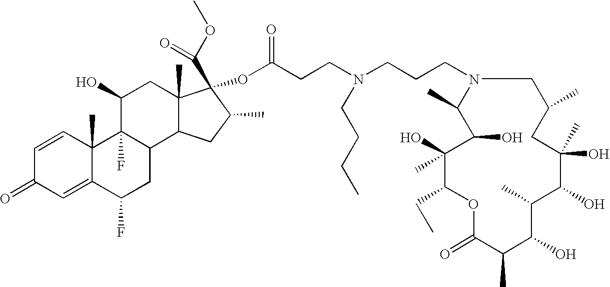

31. The method according to claim 1 whereby the compound of the formula VII has the structure:

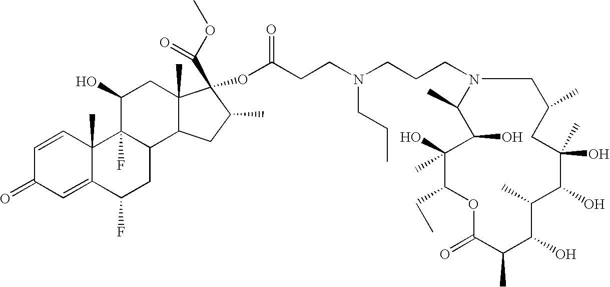

32. The method according to claim 1, whereby the inflammatory disease disorder or condition of the gastrointestinal tract is an inflammatory bowel disease.

33. The method according to claim 32, wherein the inflammatory bowel disease is Crohn's disease, ulcerative colitis or celiac disease.

34. The method of claim 1 wherein the subject has been previously determined to be responsive to treatment with an active ingredient comprising as its active moiety the subunit T.

35. A method for the maintenance treatment of an inflammatory disease, disorder or condition of the gastrointestinal tract or the delay or prevention of recurrence of said disease, disorder or condition comprising administering to a human or nonhuman mammalian subject in need thereof an effective amount of a low oral bioavailability conjugate compound of formula VII 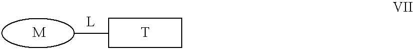
wherein M represents a group of Formula XIV: 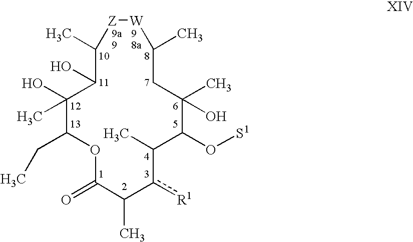
wherein
(i) Z and W are, together, 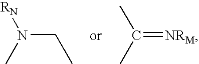
wherein
R_{M} is hydroxy, alkoxy, substituted alkoxy or OR^{p};
R_{N} is hydrogen, R^{p}, or alkyl;
(ii) R¹ is hydroxy, OR^{p} or —O—S²;
(iii) S¹ is hydrogen or a sugar moiety at position C/5 of the formula: 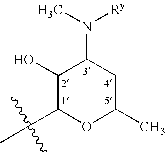
wherein R^{y} is H, alkyl, or R^{p};
(iv) S² is H or a sugar moiety of the formula: 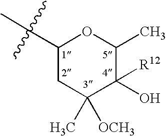
wherein
R¹² is hydrogen, alkyl, alkyl-R^{p}, or R^{p}; and
R^{p} is hydroxyl or amino protective group;
wherein M has a linkage site through which it is linked to the subunit T via the linking group L; provided that the linkage site being at one or more of the following:
a) any reactive hydroxy, nitrogen, or epoxy group located on macrolide ring, sugar moiety S¹, sugar moiety S², or an aglycone oxygen or nitrogen when S¹ and/or S² is cleaved off;
b) a reactive >N—R_{N} or 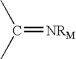
group located on Z and W; and
T is a steroidal or nonsteroidal anti-inflammatory subunit;
L is a linker molecule to which each of M and T are covalently linked, wherein L represents a group of Formula IXA or Formula IXB:
X¹—(CH₂)ₘ—X² IXA
X¹—(CH₂)ₘ-Q-(CH₂)ₙ—X² IXB
wherein
X¹ is selected from the group consisting of —CH₂—, —CH₂NH—, —C(═O)—, —OC(═O)—, ═N—O—, —OC(═O)NH— and —C(═O)NH—;
X² is —NH—, —CH₂—, —NHC(═O)—, —C(═O)—, —O— or —OC(═O)—;
Q is —NH— or —CH₂—, wherein each —CH₂— or —NH— group may be optionally substituted by C₁-C₇-alkyl, C₂-C₇-alkenyl, C₂-C₇-alkynyl, C(═O)R^{x}, C(═O)OR^{x}, C(═O)NHR^{x}, wherein R^{x} may be C₁-C₇-alkyl, aryl or heteroaryl;
m and n independently are a whole number from 0 to 8, with the proviso that if Q is NH, n cannot be 0;
and pharmaceutically acceptable salts, prodrugs, and solvates thereof in an oral dosage form; and
wherein the conjugate of formula VII exhibits less than 10% oral bioavailability.

36. The method of claim 35, wherein
wherein M represents a group of Formula XV: 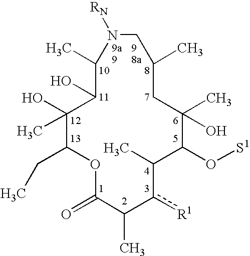
wherein
(i) R_{N} is hydrogen, R^{p}, or alkyl;
(ii) R¹ is hydroxy or —O—S²;
(iv) S¹ is hydrogen or a sugar moiety at position C/5 of the formula: 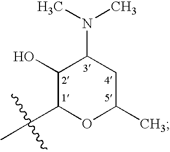
(v) S² is H or a sugar moiety of the formula: 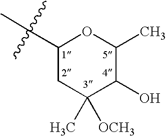
wherein R^{p} amino protective group; and
wherein M has a linkage site through which it is linked to the subunit T via the linking group L; provided that the linkage site is the reactive >N—R_{N} via the linking group L.

37. The method according to claim 8 wherein T is dexamethasone, flumetasone, or desoxymethasone.

38. The method according to claims 9 wherein T represents a NSAID subunit selected from the group consisting of: flunixin, indomethacin, celecoxib, tetrafluorthalidomide, mefenamic acid, and meclofenamic acid.

39. The method according to claim 5, wherein M is 9a-aza-9a-homoerythromycin A aglycone, and T is selected from the group consisting of flunixin, indomethacin, and flumethesone.

40. The method according to claim 5, wherein M is azithromycin or decladinosyl azithromycin; T is selected from the group consisting of flunixin and dexamethasone; and the linkage site through which M is linked to the subunit T via the linking group L at a reactive nitrogen located on S¹.

41. The method according to claim 8, wherein M is erythromycin or 3-keto decladinosyl 6-O-methyl erythromycin.
